SEQUENCE LISTING (80 sequences)

SEQ ID NO: 1 - protein, 133 aa, Homo sapiens [sequence omitted]

SEQ ID NO: 2 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 3 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 4 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - protein, 236 aa, Artificial Sequence
  source: /note=“Description of Artificial Sequence Synthetic polypeptide”
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 8 - protein, 236 aa, Artificial Sequence
  source: /note=“Description of Artificial Sequence Synthetic polypeptide”
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 9 - protein, 241 aa, Artificial Sequence
  source: /note=“Description of Artificial Sequence Synthetic polypeptide”
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 10 - protein, 241 aa, Artificial Sequence
  source: /note=“Description of Artificial Sequence Synthetic polypeptide”
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 11 - protein, 246 aa, Artificial Sequence
  source: /note=“Description of Artificial Sequence Synthetic polypeptide”
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 12 - protein, 246 aa, Artificial Sequence
  source: /note=“Description of Artificial Sequence Synthetic polypeptide”
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 13 - protein, 521 aa, Artificial Sequence
  source: /note=“Description of Artificial Sequence Synthetic polypeptide”
RTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 14 - protein, 521 aa, Artificial Sequence; identical to SEQ ID NO: 13
  source: /note=“Description of Artificial Sequence Synthetic polypeptide”

SEQ ID NO: 15 - protein, 210 aa, Artificial Sequence
  source: /note=“Description of Artificial Sequence Synthetic polypeptide”
RTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 16 - protein, 312 aa, Artificial Sequence
  source: /note=“Description of Artificial Sequence Synthetic polypeptide”
RTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 17 - protein, 599 aa, Artificial Sequence
  source: /note=“Description of Artificial Sequence Synthetic polypeptide”
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 18 - protein, 198 aa, Artificial Sequence
  source: /note=“Description of Artificial Sequence Synthetic polypeptide”
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDI

SEQ ID NO: 19 - protein, 300 aa, Artificial Sequence
  source: /note=“Description of Artificial Sequence Synthetic polypeptide”
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 20 - protein, 760 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - protein, 705 aa, Artificial Sequence
  source: /note=“Description of Artificial Sequence Synthetic polypeptide”
  SITE (451)..(470): /note=“This region may encompass 1-4 ‘Gly Gly Gly Gly Ser’ repeating units”
  SITE (488)..(507): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDI

SEQ ID NO: 22 - protein, 909 aa, Artificial Sequence
  source: /note=“Description of Artificial Sequence Synthetic polypeptide”
  SITE (553)..(572): /note=“This region may encompass 1-4 ‘Gly Gly Gly Gly Ser’ repeating units”
  SITE (590)..(609): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPAGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 23 - protein, 718 aa, Artificial Sequence
  source: /note=“Description of Artificial Sequence Synthetic polypeptide”
  SITE (464)..(483): /note=“This region may encompass 1-4 ‘Gly Gly Gly Gly Ser’ repeating units”
  SITE (501)..(520): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDI

SEQ ID NO: 24 - protein, 922 aa, Artificial Sequence
  source: /note=“Description of Artificial Sequence Synthetic polypeptide”
  SITE (566)..(585): /note=“This region may encompass 1-4 ‘Gly Gly Gly Gly Ser’ repeating units”
  SITE (603)..(622): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPAGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 25 - protein, 838 aa, Artificial Sequence
  source: /note=“Description of Artificial Sequence Synthetic polypeptide”
  SITE (482)..(501): /note=“This region may encompass 1-4 ‘Gly Gly Gly Gly Ser’ repeating units”
  SITE (519)..(538): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 26 - protein, 851 aa, Artificial Sequence
  source: /note=“Description of Artificial Sequence Synthetic polypeptide”
  SITE (495)..(514): /note=“This region may encompass 1-4 ‘Gly Gly Gly Gly Ser’ repeating units”
  SITE (532)..(551): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 27 - protein, 838 aa, Artificial Sequence
  source: /note=“Description of Artificial Sequence Synthetic polypeptide”
  SITE (482)..(501): /note=“This region may encompass 1-4 ‘Gly Gly Gly Gly Ser’ repeating units”
  SITE (519)..(538): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 28 - protein, 851 aa, Artificial Sequence
  source: /note=“Description of Artificial Sequence Synthetic polypeptide”
  SITE (495)..(514): /note=“This region may encompass 1-4 ‘Gly Gly Gly Gly Ser’ repeating units”
  SITE (532)..(551): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 29 - protein, 5 aa, Artificial Sequence
  source: /note=“Description of Artificial Sequence Synthetic peptide”
GGGGS

SEQ ID NO: 30 - protein, 10 aa, Artificial Sequence
  source: /note=“Description of Artificial Sequence Synthetic peptide”
GGGGSGGGGS

SEQ ID NO: 31 - protein, 15 aa, Artificial Sequence
  source: /note=“Description of Artificial Sequence Synthetic peptide”
GGGGSGGGGSGGGGS

SEQ ID NO: 32 - protein, 20 aa, Artificial Sequence
  source: /note=“Description of Artificial Sequence Synthetic peptide”
  VARIANT (10)..(10): /replace=“Asn”
  SITE (1)..(20): /note=“Variant residues given in the sequence have no preference with respect to those in the annotations for variant positions”
GGGGSGGGGAGGGGSGGGGS

SEQ ID NO: 33 - protein, 60 aa, Artificial Sequence
  source: /note=“Description of Artificial Sequence Synthetic polypeptide”
  VARIANT (10)..(10): /replace=“Ala” or “Asn”
  VARIANT (15)..(15): /replace=“Asn”
  VARIANT (30)..(30): /replace=“Ala” or “Asn”
  VARIANT (35)..(35): /replace=“Asn”
  VARIANT (50)..(50): /replace=“Ala” or “Asn”
  VARIANT (55)..(55): /replace=“Asn”
  SITE (1)..(60): /note=“This sequence may encompass 1-3 ‘Gly Gly Gly Gly Ser Gly Gly Gly Gly Xaa Gly Gly Gly Gly Xaa Gly Gly Gly Gly Ser’ repeating units”
  SITE (1)..(60): /note=“Variant residues given in the sequence have no preference with respect to those in the annotations for variant positions”
GGGGSGGGGSGGGGAGGGGSGGGGSGGGGSGGGGAGGGGSGGGGSGGGGSGGGGAGGGGS

SEQ ID NO: 34 - protein, 16 aa, Artificial Sequence
  source: /note=“Description of Artificial Sequence Synthetic peptide”
GGSLSGRSDNHGGGGS

SEQ ID NO: 35 - protein, 13 aa, Artificial Sequence
  source: /note=“Description of Artificial Sequence Synthetic peptide”
GGSLSGRSDNHGS

SEQ ID NO: 36 - protein, 42 aa, Artificial Sequence
  source: /note=“Description of Artificial Sequence Synthetic polypeptide”
GGGGSGGGGSGGGGSISSGLLSSGGSGGSLSGRSDNHGGGGS

SEQ ID NO: 37 - protein, 6 aa, Artificial Sequence
  source: /note=“Description of Artificial Sequence Synthetic peptide”
GPLGVR

SEQ ID NO: 38 - protein, 7 aa, Artificial Sequence
  source: /note=“Description of Artificial Sequence Synthetic peptide”
PLGMWSR

SEQ ID NO: 39 - protein, 7 aa, Artificial Sequence
  source: /note=“Description of Artificial Sequence Synthetic peptide”
PLGLWAR

SEQ ID NO: 40 - protein, 8 aa, Artificial Sequence
  source: /note=“Description of Artificial Sequence Synthetic peptide”
PQGIAGQR

SEQ ID NO: 41 - protein, 6 aa, Artificial Sequence
  source: /note=“Description of Artificial Sequence Synthetic peptide”
PLGLAG

SEQ ID NO: 42 - protein, 6 aa, Artificial Sequence
  source: /note=“Description of Artificial Sequence Synthetic peptide”
LALGPR

SEQ ID NO: 43 - protein, 10 aa, Artificial Sequence
  source: /note=“Description of Artificial Sequence Synthetic peptide”
GGPLGMLSQS

SEQ ID NO: 44 - protein, 9 aa, Artificial Sequence
  source: /note=“Description of Artificial Sequence Synthetic peptide”
GGGGRRGGS

SEQ ID NO: 45 - protein, 8 aa, Artificial Sequence
  source: /note=“Description of Artificial Sequence Synthetic peptide”
TGRGPSWV

SEQ ID NO: 46 - protein, 8 aa, Artificial Sequence
  source: /note=“Description of Artificial Sequence Synthetic peptide”
SARGPSRW

SEQ ID NO: 47 - protein, 8 aa, Artificial Sequence
  source: /note=“Description of Artificial Sequence Synthetic peptide”
TARGPSFK

SEQ ID NO: 48 - protein, 7 aa, Artificial Sequence
  source: /note=“Description of Artificial Sequence Synthetic peptide”
TARGPSW

SEQ ID NO: 49 - protein, 8 aa, Artificial Sequence
  source: /note=“Description of Artificial Sequence Synthetic peptide”
GGWHTGRN

SEQ ID NO: 50 - protein, 8 aa, Artificial Sequence
  source: /note=“Description of Artificial Sequence Synthetic peptide”
HTGRSGAL

SEQ ID NO: 51 - protein, 8 aa, Artificial Sequence
  source: /note=“Description of Artificial Sequence Synthetic peptide”
PLTGRSGG

SEQ ID NO: 52 - protein, 7 aa, Artificial Sequence
  source: /note=“Description of Artificial Sequence Synthetic peptide”
LTGRSGA

SEQ ID NO: 53 - protein, 8 aa, Artificial Sequence
  source: /note=“Description of Artificial Sequence Synthetic peptide”
RQARVVNG

SEQ ID NO: 54 - protein, 18 aa, Artificial Sequence
  source: /note=“Description of Artificial Sequence Synthetic peptide”
VHMPLGFLGPRQARVVNG

SEQ ID NO: 55 - protein, 795 aa, Artificial Sequence
  source: /note=“Description of Artificial Sequence Synthetic polypeptide”
  SITE (439)..(458): /note=“This region may encompass 1-4 ‘Gly Gly Gly Gly Ser’ repeating units”
  SITE (476)..(495): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 56 - protein, 875 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 57 - protein, 887 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSTGRGPSWVGGGGSGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 58 - protein, 816 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSTGRGPSWVGGGGSGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 59 - protein, 816 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSTGRGPSWVGGGGSGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 60 - protein, 904 aa, Artificial Sequence
  source: /note=“Description of Artificial Sequence Synthetic polypeptide”
  SITE (548)..(567): /note=“This region may encompass 1-4 ‘Gly Gly Gly Gly Ser’ repeating units”
  SITE (585)..(604): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSVHMPLGFLGPRQARVVNGGGGSGGGGSGGGGSGGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTG

SEQ ID NO: 61 - protein, 766 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 62 - protein, 778 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSTGRGPSWVGGGGSGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 63 - protein, 707 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSTGRGPSWVGGGGSGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 64 - protein, 707 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSTGRGPSWVGGGGSGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 65 - protein, 780 aa, Artificial Sequence
  source: /note=“Description of Artificial Sequence Synthetic polypeptide”
  SITE (228)..(252): /note=“This region may encompass 2-5 ‘Gly Gly Gly Gly Ser’ repeating units”
  SITE (450)..(474): /note=“This region may encompass 2-5 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 66 - protein, 705 aa, Artificial Sequence
  source: /note=“Description of Artificial Sequence Synthetic polypeptide”
  SITE (439)..(458): /note=“This region may encompass 1-4 ‘Gly Gly Gly Gly Ser’ repeating units”
  SITE (476)..(495): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIGGGGSGGGGSGGGGSGGGGSVHMPLGFLGPRQARVVNGGGGSGGGGSGGGGSGGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 67 - protein, 909 aa, Artificial Sequence
  source: /note=“Description of Artificial Sequence Synthetic polypeptide”
  SITE (541)..(560): /note=“This region may encompass 1-4 ‘Gly Gly Gly Gly Ser’ repeating units”
  SITE (578)..(597): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGGGGGSGGGGSGGGGSGGGGSVHMPLGFLGPRQARVVNGGGGSGGGGSGGGGSGGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 68 - protein, 718 aa, Artificial Sequence
  source: /note=“Description of Artificial Sequence Synthetic polypeptide”
  SITE (452)..(471): /note=“This region may encompass 1-4 ‘Gly Gly Gly Gly Ser’ repeating units”
  SITE (489)..(508): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIGGGGSGGGGSGGGGSGGGGSVHMPLGFLGPRQARVVNGGGGSGGGGSGGGGSGGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 69 - protein, 922 aa, Artificial Sequence
  source: /note=“Description of Artificial Sequence Synthetic polypeptide”
  SITE (554)..(573): /note=“This region may encompass 1-4 ‘Gly Gly Gly Gly Ser’ repeating units”
  SITE (591)..(610): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGGGGGSGGGGSGGGGSGGGGSVHMPLGFLGPRQARVVNGGGGSGGGGSGGGGSGGGGSRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPA

SEQ ID NO: 70 - protein, 838 aa, Artificial Sequence
  source: /note=“Description of Artificial Sequence Synthetic polypeptide”
  SITE (541)..(560): /note=“This region may encompass 1-4 ‘Gly Gly Gly Gly Ser’ repeating units”
  SITE (578)..(597): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGGGGGSGGGGSGGGGSGGGGSVHMPLGFLGPRQARVVNGGGGSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 71 - protein, 851 aa, Artificial Sequence
  source: /note=“Description of Artificial Sequence Synthetic polypeptide”
  SITE (554)..(573): /note=“This region may encompass 1-4 ‘Gly Gly Gly Gly Ser’ repeating units”
  SITE (591)..(610): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGGGGGSGGGGSGGGGSGGGGSVHMPLGFLGPRQARVVNGGGGSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 72 - protein, 838 aa, Artificial Sequence
  source: /note=“Description of Artificial Sequence Synthetic polypeptide”
  SITE (541)..(560): /note=“This region may encompass 1-4 ‘Gly Gly Gly Gly Ser’ repeating units”
  SITE (578)..(597): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGGGGGSGGGGSGGGGSGGGGSVHMPLGFLGPRQARVVNGGGGSGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 73 - protein, 851 aa, Artificial Sequence
  source: /note=“Description of Artificial Sequence Synthetic polypeptide”
  SITE (554)..(573): /note=“This region may encompass 1-4 ‘Gly Gly Gly Gly Ser’ repeating units”
  SITE (591)..(610): /note=“This region may encompass 1-4 ‘Gly Gly Gly Gly Ser’ repeating units”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSTGRGPSWVGGGGSGGGSKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGGGGGSGGGGSGGGGSGGGGSVHMPLGFLGPRQARVVNGGGGSGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 74 - protein, 133 aa, Artificial Sequence
  source: /note=“Description of Artificial Sequence Synthetic polypeptide”
  VARIANT (3)..(3): /replace=“Ala”
  VARIANT (20)..(20): /replace=“His” or “Lys” or “Leu” or “Met” or “Asn” or “Gln” or “Arg” or “Ser” or “Val” or “Tyr”
  VARIANT (42)..(42): /replace=“Gly” or “Ser” or “Thr” or “Gln” or “Glu” or “Asn” or “Asp” or “Arg” or “Lys”
  VARIANT (45)..(45): /replace=“Gly” or “Ser” or “Thr” or “Gln” or “Glu” or “Asn” or “Asp” or “Arg” or “Lys”
  VARIANT (72)..(72): /replace=“Gly” or “Ser” or “Thr” or “Gln” or “Glu” or “Asn” or “Asp” or “Arg” or “Lys”
  VARIANT (88)..(88): /replace=“Ala” or “Glu” or “Phe” or “His” or “Lys” or “Leu” or “Met” or “Ser” or “Thr” or “Val” or “Trp” or “Tyr”
  VARIANT (90)..(90): /replace=“Thr”
  VARIANT (125)..(125): /replace=“Ser”
  VARIANT (126)..(126): /replace=“Asp” or “Phe” or “Gly” or “His” or “Ile” or “Lys” or “Leu” or “Pro” or “Ser” or “Thr” or “Trp” or “Tyr”
  SITE (1)..(133): /note=“Variant residues given in the sequence have no preference with respect to those in the annotations for variant positions”
  source: /note=“See specification as filed for detailed description of substitutions and preferred embodiments”
APNSSSTKKTQLQLEHLLLDLQMILNGINNYKNPKLTRMLTAKFAMPKKATELKHLQCLEEELKPLEEVLNAAQSKNFHLRPRDLISNINVIVLELKGSETTFMCEYADETATIVEFLNRWITFCASIISTLT

SEQ ID NO: 75 - protein, 133 aa, Artificial Sequence
  source: /note=“Description of Artificial Sequence Synthetic polypeptide”
  VARIANT (3)..(3): /replace=“Ala”
  VARIANT (20)..(20): /replace=“His” or “Lys” or “Leu” or “Met” or “Asn” or “Gln” or “Arg” or “Ser” or “Val” or “Tyr”
  VARIANT (38)..(38): /replace=“Lys” or “Ser”
  VARIANT (42)..(42): /replace=“Gly” or “Ile” or “Ser” or “Thr” or “Gln” or “Glu” or “Asn” or “Asp” or “Arg” or “Lys”
  VARIANT (45)..(45): /replace=“Gly” or “Ser” or “Thr” or “Gln” or “Glu” or “Asn” or “Asp” or “Arg” or “Lys”
  VARIANT (62)..(62): /replace=“Leu” or “Ala” or “Ile”
  VARIANT (68)..(68): /replace=“Val”
  VARIANT (73)..(73): /replace=“Thr”
  VARIANT (88)..(88): /replace=“Ala” or “Glu” or “Phe” or “His” or “Lys” or “Leu” or “Met” or “Ser” or “Thr” or “Val” or “Trp” or “Tyr”
  VARIANT (90)..(90): /replace=“Thr”
  VARIANT (125)..(125): /replace=“Ser”
  VARIANT (126)..(126): /replace=“Asp” or “Phe” or “Gly” or “His” or “Ile” or “Lys” or “Leu” or “Pro” or “Ser” or “Thr” or “Trp” or “Tyr”
  SITE (1)..(133): /note=“Variant residues given in the sequence have no preference with respect to those in the annotations for variant positions”
  source: /note=“See specification as filed for detailed description of substitutions and preferred embodiments”
APNSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCASIISTLT

SEQ ID NO: 76 - protein, 484 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGPLGVRGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 77 - protein, 489 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGPLGVRGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 78 - protein, 489 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGPLGVRGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 79 - protein, 494 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGPLGVRGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKGGGGSGGGGSGGGGSGGGGSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 80 - protein, 20 aa, Artificial Sequence
  source: /note=“Description of Artificial Sequence Synthetic peptide”
GGGGSGGGGSGGGGSGGGGS

(19) United States
(12) Patent Application Publication
(10) Pub. No.: US 2022/0356221 A1
(43) Pub. Date: Nov. 10, 2022

(54) CYTOKINE PRODRUGS AND DUAL-PRODRUGS

(71) Applicant: ASKGENE PHARMA, INC., Camarillo, CA (US)

(72) Inventors: Yuefeng LU, Moorpark, CA (US); Chunxiao YU, Santa Barbara, CA (US)

(73) Assignee: ASKGENE PHARMA, INC., Camarillo, CA (US)

(21) Appl. No.: 17/762,864

(22) PCT Filed: Sep. 28, 2020
(86) PCT No.: PCT/US2020/053155

§ 371 Date: Mar. 23, 2022

Related U.S. Application Data
(60) Provisional application No. 62/907,615, filed on Sep. 28, 2019.

(51) Int. Cl. C07K 14/54; C07K 16/24; C07K 14/715; A61P 35/00; A61K 45/06; A61K 38/20; A61K 39/395; A61K 38/17

(52) U.S. Cl. CPC C07K 14/5434; C07K 16/244; C07K 14/7155; A61P 35/00; A61K 45/06; A61K 38/208; A61K 39/3955; A61K 38/1793; C07K 2319/00; C07K 2317/622; C07K 2317/55; C07K 2317/76; C07K 2319/30; A61K 2039/505

(57) ABSTRACT

The present application relates to novel prodrugs of IL-12. Further included in the present application are methods of making and using the novel prodrugs.

### CROSS REFERENCE TO RELATED APPLICATION

The present application claims priority from U.S. Provisional Application No. 62/907,615, filed on Sep. 28, 2019, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 23, 2020, is named 025471_WO007_SL.txt and is 282,851 bytes in size.

### BACKGROUND OF THE INVENTION

Interleukin 12 (IL-12) plays important roles in immunity (Watford et al., Cytokine Growth Factor Rev. (2003) 14(5):361-8). It stimulates interferon gamma release and activates T cells and natural killer (NK) cells (Del Vecchio, Clin Cancer Res. (2007) 13(16):4677-85). IL-12 is comprised of two subunits, p40 and p35. IL-12 receptor consists of two subunits, IL-12 receptor beta 1 (IL-12Rβ1) and beta 2 (IL-12Rβ2). Deficiency in IL-12Rβ1 impairs the IL-12 signal pathway and may lead to bacterial infection and even autoimmunity such as Sjögren's Syndrome (Sogkas et al., Front Immunol. (2017) 8:885). IL-12 has previously been explored as a potential therapy to treat cancer (Colombo and Trinchieri, Cytokine Growth Factor Rev. (2002) 13(2):155-68; Del Vecchio, supra; Lasek et al., Cancer Immunol Immunother. (2014) 63(5):419-35). Several clinical trials on IL-12 therapy showed limited efficacy, potentially due to its narrow therapeutic index (Lasek et al., supra).

IL-2, another important cytokine, plays a central role in lymphocyte generation, survival and homeostasis. It has 133 amino acids and consists of four antiparallel, amphiphatic alpha-helices that form a quaternary structure indispensable of its function (Smith, Science (1988) 240:1169-76; Bazan, Science (1992) 257:410-13).

IL-2 acts by binding to IL-2 receptors (IL-2R), which consist of up to three individual subunits. Association of the α (CD25), β (CD122), and γ (CD132) subunits results in a trimeric, high-affinity IL-2R. Dimeric IL-2R consisting of the β and γ subunits is termed intermediate-affinity IL-2R. The a subunit forms the monomeric low affinity IL-2R. Although the dimeric intermediate-affinity IL-2 receptor binds IL-2 with approximately 100-fold lower affinity than the trimeric high-affinity receptor, both the dimeric and trimeric IL-2R variants are able to transmit signal upon IL-2 binding (Minami et al, Annu Rev Immunol. (1993) 11:245-68). Hence, the a subunit is not essential for IL-2 signaling, but confers high-affinity binding to an IL-2R, whereas the β and γ subunit are crucial for signal transduction (Krieg et al., Proc Natl Acad Sci. (2010) 107:11906-11). Trimeric IL-2R is expressed by CD4⁺ FoxP3⁺ regulatory T (Treg) cells. They are also transiently induced on conventional activated T cells, whereas in the resting state these cells express only dimeric IL-2R. Treg cells consistently express the highest level of CD25 in vivo (Fontenot et al., Nature Immunol. (2005) 6:1142-51).

Muteins of IL-2 have been made to reduce the side effects of IL-2. See, e.g., U.S. Pat. Nos. 6,955,807 and 9,428,567 and EP2639241B1. Pegylated IL-2 with reduced affinity to the high-affinity trimeric IL-2R has also been disclosed (US20140328791). In addition, IL-2 muteins have been fused to antibodies, such as PD-L1 antibody (e.g., WO 2017/220989) and CEA antibody (e.g., Klein et al., Oncoimmunology (2017) 6(3):e1277306; U.S. Pat. No. 9,206,260). The CEA antibody was maturated from an original mouse CEA antibody PR1A3 (U.S. Pat. No. 8,642,742). While the antibodies may target the IL-2 muteins to the tumor site, the amount of antibody-IL-2 fusion at the tumor site is likely only a small portion of the administered dose, with a large part of the administered dose still in the circulation. The circulating IL-2 would bind to the so-called “PK sinkers” such as IL-2Rs on immune cells localized in immune organs, e.g., lymph nodes and the spleen. Therefore, even with the tumor-targeting specificity of the antibody, the total amount of the IL-2 that can be dosed would still be limited.

IL-15 is a cytokine with structural similarities to IL-2. IL-15 is secreted by mononuclear phagocytes and other immune cells following viral infection. IL-15 induces proliferation of NK and other immune cells and is involved in the killing of virally infected cells and cancer cells. Like IL-2, IL-15 binds to the IL-2R β/γ complex, the intermediate-affinity receptor, with a K_{D} of about 1 nM (Giri et al., EMBO J. (1994) 13:2822-30). IL-15 binds to IL-15 receptor (IL-15R) α with a much higher affinity (K_{D}˜0.05 nM). IL-15Rα can associate with the IL-2Rβ/γ complex to form an IL-15-specific, functional high-affinity receptor (αβγ) (Minami et al., Annu Rev Immunol. (1993) 11:245-67; Giri et al., J Leukoc Biol. (1995) 5745:763-6; and Lehours et al., Eur Cytokine Netw. (2000) 11:207-15). The extracellular region of IL-15Rα contains a Sushi domain, which is a common motif in protein-protein interaction. It has been shown that an IL-15Ra N-terminal fragment with the first 65 amino acids is partially active, while the fragment with the first 85 amino acids is fully functional (Wei et al., J Immunol. (2001) 167(1):277-82).

Unfortunately, the adverse effects of the current cytokine drug candidates are significant, limiting the dosing amounts of such drugs. In addition, the activation of T, NK, and other immune cells by these drug candidates is not site-specific. Further, there appears to be “PK sinkers” for cytokine muteins as well, even when the muteins' affinities for the cytokine receptors have been significantly reduced. There are also numerous difficulties in the production of cytokine-based protein therapeutics.

Hence, there is a need to develop improved cytokine-based cancer therapeutics that are more tumor site-selective, avoid PK sinkers, and have fewer severe side effects while having improved efficacy.

### SUMMARY OF THE INVENTION

The present disclosure provides an IL-12 prodrug or (“prodrug”) comprising an IL-12 cytokine moiety, a masking moiety (M), a dimeric carrier moiety (C), and at least one peptide linker, wherein the IL-12 cytokine moiety comprises an IL-12 p40 subunit (p40) and an IL-12 p35 subunit (p35), the masking moiety binds to the IL-12 cytokine moiety and inhibits a biological activity of the IL-12 cytokine moiety, and the masking moiety is fused to one polypeptide chain of the dimeric carrier moiety, optionally via a peptide linker, and the IL-12 cytokine moiety is fused to the other polypeptide chain of the carrier moiety, optionally via a peptide linker, or the p40 subunit is fused to one polypeptide chain of the carrier moiety, optionally via a peptide linker, the p35 subunit is fused to the other polypeptide chain of the carrier moiety, optionally via a peptide linker, and the masking moiety is fused to the C-terminus of the p40 subunit or the p35 subunit, optionally via a peptide linker.

In particular embodiments, the IL-12 subunit p40 has an amino acid sequence at least 95% identical to that of SEQ ID NO: 5. In particular embodiments, the IL-12 subunit p35 has an amino acid sequence at least 95% identical to that of SEQ ID NO: 6.

In some embodiments, at least one peptide linker is a cleavable peptide linker. In other embodiments, at least one peptide linker is a non-cleavable peptide linker.

In some embodiments, the masking moiety is a first masking moiety (MM1) that is fused to the carrier moiety or to the IL-12 p40 or p35 subunit, optionally via a cleavable peptide linker. In particular embodiments, the first masking moiety (MM1) binds to IL-12 p40 subunit and reduces or inhibits the binding of IL-12 to IL-12 receptor 31 (IL-12Rβ1). In some embodiments, the masking moiety is selected from the extracellular domain of IL-12 receptor 31 (IL-12Rβ1 ECD) or a fragment thereof, a p40-binding peptide discovered from screening of a peptide library, and a single chain Fv (scFv) or Fab domain of an antibody that binds to p40. In other embodiments, the masking moiety is a scFv or Fab domain of an antibody that binds to p40. In particular embodiments, the masking moiety comprises a scFv that binds to p40, wherein the scFv comprises an amino acid sequence at least 99% identical to SEQ ID NOs: 7-11 and 12.

In some embodiments, the prodrug further comprises a second masking moiety (MM2) that is fused to the carrier moiety or to the IL-12 p40 or p35 subunit, optionally via a cleavable peptide linker. In particular embodiments, the second masking moiety (MM2) binds to p35 and/or p40 and inhibits the binding of IL-12 to IL-12 receptor 32 (IL-12Rβ2). In some embodiments, the second masking moiety is selected from the extracellular domain of IL-12 receptor 32 (IL-12Rβ2 ECD) or a fragment thereof, a p35 or p40-binding peptide discovered from screening of a peptide library, and a single chain Fv or Fab domain of an antibody that binds to p35 or p40 and reduces or inhibits the binding of IL-12 to IL-12Rβ2. In particular embodiments, the second masking moiety comprises the extracellular domain (ECD) of IL-12 receptor beta2 (IL-12Rβ2) or a fragment thereof, wherein the IL-12Rβ2 ECD or a fragment thereof comprises an amino acid sequence at least 95% identical to SEQ ID NO: 17, 18, or 19.

In some embodiments, the second masking moiety comprises a single chain Fv (scFv) or Fab domain of an antibody that binds to p40 and reduces or inhibits the binding of IL-12 to IL-12Rβ2; wherein said scFv of Fab comprises the same light chain CDRs and heavy chain CDRs as derived from IL-12 antibody PMA204, Antibody 1, Antibody 50, Antibody 68, Antibody 80, or Antibody 136.

In some embodiments, the carrier is an antibody or an antigen-binding moiety that binds to a target expressed on the surface of a cancer cell. In particular embodiments, the antibody or antigen-binding moiety binds to an antigen selected from 5T4, Claudin 18.2, EGFR, EGFR type III, GPC3, TROP-2, mesothelin, PSMA, CMET, DLL-3, and BCMA.

In some embodiments, the carrier moiety is selected from an Fc domain and an antibody. In particular embodiments, the carrier moiety comprises an Fc domain, which comprises a first and a second polypeptide chain, and wherein the polypeptide chains of the prodrug comprise molecular formulae (from N-terminus to C-terminus) selected from one of the following pairs:

a. F1-PL1-A-PL2-B, F2-CL1-MM2-CL2-MM1; and

b. F1-PL1-B-PL2-A, F2-CL1-MM1-CL2-MM2;

wherein F1 and F2 are subunits of the carrier moiety (e.g., Fc domain), which form a heterodimer; PL1 and PL2 are peptide linkers; CL1 and CL2 are cleavable peptide linkers; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In particular embodiments, the carrier moiety comprises an Fc domain, which comprises a first and a second polypeptide chain, wherein the polypeptide chains of the prodrug comprise molecular formulae (from N-terminus to C-terminus) selected from one of the following pairs:

a. F1-PL1-A-PL2-B, F2-PL3-MM2-CL-MM1; and

b. F1-PL1-B-PL2-A, F2-PL3-MM1-CL-MM2;

wherein F1 and F2 are subunits of the carrier moiety (e.g., Fc domain), which form a heterodimer; PL1, PL2, and PL3 are peptide linkers; CL is a cleavable peptide linker; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In particular embodiments, the carrier moiety comprises an Fc domain, which comprises a first and a second polypeptide chain, wherein the polypeptide chains of the prodrug comprise molecular formulae (from N-terminus to C-terminus) selected from one of the following pairs:

a. F1-PL1-A-CL1-MM2, F2-PL2-B-CL2-MM1; and

b. F1-PL1-A-CL1-MM1, F2-PL2-B-CL2-MM2;

wherein F1 and F2 are subunits of the carrier moiety (e.g., Fc domain), which form a heterodimer; PL1 and PL2 are peptide linkers; CL1 and CL2 are cleavable peptide linkers; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In particular embodiments, the carrier moiety comprises an Fc domain, which comprises a first and a second polypeptide chain, wherein the polypeptide chains comprise molecular formulae (from N-terminus to C-terminus) selected from one of the following pairs:

a. F1-PL1-A-CL-MM2, F2-PL2-B-PL3-MM1; and

b. F1-PL1-A-PL2-MM1, F2-PL3-B-CL-MM2;

wherein F1 and F2 are subunits of the carrier moiety (e.g., Fc domain), which form a heterodimer; PL1, PL2, and PL3 are peptide linkers; CL is a cleavable peptide linker; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In particular embodiments, the carrier moiety comprises an Fc domain, which comprises a first and a second polypeptide chain, wherein the polypeptide chains comprise molecular formulae (from N-terminus to C-terminus) selected from one of the following pairs:

a. F1-PL1-A-PL2-B, F2-CL-M; and

b. F1-PL1-B-PL2-A, F2-CL-M;

wherein F1 and F2 are subunits of the carrier moiety (e.g., Fc domain), which form a heterodimer; PL1 and PL2 are peptide linkers; CL is a cleavable peptide linker; M is a masking moiety that binds to p40 subunit and inhibits a biological activity of IL-12; A is an IL-12 p35 subunit; and B is an IL-12 p40 subunit.

In particular embodiments, the carrier moiety comprises an Fe domain, which comprises a first and a second polypeptide chain, wherein the polypeptide chains comprise molecular formulae (from N-terminus to C-terminus) selected from one of the following pairs:

a. F1-PL1-A-PL2-M, F2-PL3-B; and

b. F1-PL1-B-PL2-A, F2-PL3-M;

wherein F1 and F2 are subunits of the carrier moiety (e.g., Fc domain), which form a heterodimer; PL1, PL2, and PL3 are peptide linkers; M is a masking moiety that binds to p40 subunit and inhibits a biological activity of IL-12; A is an IL-12 p35 subunit; and B is an IL-12 p40 subunit.

In particular embodiments, the carrier moiety comprises an Fc domain, which comprises a first and a second polypeptide chain, wherein the polypeptide chains comprise molecular formulae (from N-terminus to C-terminus) selected from one of the following pairs:

a. F1-PL1-M, F2-PL2-A-SS-B; and

b. F1-CL, F2-PL1-A-SS-B;

wherein F1 and F2 are subunits of the carrier moiety (e.g., Fc domain), which form a heterodimer; PL1 and PL2 are peptide linkers; M is a masking moiety that binds to p40 subunit and inhibits a biological activity of IL-12; A is an IL-12 p35 subunit; and B is an IL-12 p40 subunit; and SS is a disulfide bond.

In some embodiments, embodiments, the non-cleavable peptide linker comprises an amino acid sequence from SEQ ID NOs: 29-33; and wherein the cleavable peptide linker comprises an amino acid sequence selected from SEQ ID NOs: 34-54.

In some embodiments, the prodrug comprises two polypeptide chains, wherein the first polypeptide chain comprises an amino acid sequence at least 99% identical to SEQ ID NO: 20; and wherein the second polypeptide chain comprises an amino acid sequence at least 99% identical to one selected from SEQ ID NOs: 21-28.

In some embodiments, the prodrug comprises two polypeptide chains, wherein the first polypeptide chain comprises an amino acid sequence at least 99% identical to SEQ ID NO: 55; and wherein the second polypeptide chain comprises an amino acid sequence at least 99% identical to one selected from SEQ ID NOs: 56-59.

In some embodiments, the prodrug comprises two polypeptide chains, wherein the first polypeptide chain comprises an amino acid sequence at least 99% identical to SEQ ID NO: 60; and the second polypeptide chain comprises an amino acid sequence at least 99% identical to one selected from SEQ ID NOs: 61-64.

In some embodiments, the prodrug wherein the prodrug comprises two polypeptide chains, wherein the first polypeptide chain comprises an amino acid sequence at least 99% identical to SEQ ID NO: 65; and the second polypeptide chain comprises an amino acid sequence at least 99% identical to one selected from SEQ ID NOs: 66-73.

In some embodiments, the prodrug comprises two polypeptide chains, wherein the first polypeptide chain comprises an amino acid sequence at least 99% identical to SEQ ID NO: 20; and the second polypeptide chain comprises an amino acid sequence at least 99% identical to one selected from SEQ ID NOs: 76-79.

In some embodiments, the present disclosure provides a pharmaceutical composition comprising the present prodrugs and a pharmaceutically acceptable excipient.

In some aspects, the present disclosure provides a polynucleotide or polynucleotides encoding the present prodrugs, expression vectors comprising the polynucleotides, and host cells (e.g., mammalian host cells such as CHO, NS0 cells, and 293T cells) comprising the expression vectors. In some embodiments, a host cell described herein has the gene or genes encoding uPA, MMP-2, MMP-9 and/or matriptase knocked out. The present disclosure also provides methods of making the present prodrugs, comprising culturing the host cells under conditions that allow expression of prodrugs and isolating (also purifying) the prodrugs.

The present disclosure also provides a method of treating a cancer or an infectious disease or stimulating the immune system in a patient in need thereof, comprising administering to the patient a therapeutically effective amount of the prodrug or the pharmaceutical composition of the present disclosure. The present disclosure also provides a method of treating cancer comprising administering to a cancer patient the prodrug or pharmaceutical composition of the present disclosure in combination with a second pharmaceutical composition, wherein the second pharmaceutical composition comprises an active ingredient selected from a cytokine other than IL-12 or its fusion molecule, an antibody against PD-1, an antibody against PD-L1, an antibody against CTLA-4, an antibody against CD47, a PD-1 antibody-IL-2 fusion molecule, a PD-1-IL-7 fusion molecule, a PD-1 antibody-IL-15 fusion molecule, and a PD-1-IL-21 fusion molecule. The patient may have, for example, a viral infection (e.g., HIV, HBV, HCV, or HPV infection), or a cancer selected from the group consisting of brain cancer, breast cancer, lung cancer, pancreatic cancer, esophageal cancer, medullary thyroid cancer, ovarian cancer, uterine cancer, prostate cancer, testicular cancer, colorectal cancer, and stomach cancer.

Also provided herein are prodrugs for use in treating a cancer or an infectious disease or stimulating the immune system in the present method; use of an I1-12 prodrug for the manufacture of a medicament for treating a cancer or an infectious disease or stimulating the immune system in the present method; and articles of manufacture (e.g., kits) comprising one or more dosing units of the present I1-12 prodrug.

### BRIEF DESCRIPTION OF THE DRAWINGS

FIGS. 1A and 1B show an illustration of the structure of an IL-12 prodrug comprising an IL-12 p40 subunit (p40), an IL-12 p35 subunit (p35), a first masking moiety (MM1; white box), a second masking moiety (MM2; black box), a carrier (C), and two cleavable peptide linkers. FIG. 1A shows the IL-12 subunit p35 fused to the C-terminus of one of the Fc domain polypeptides, while the IL-12 subunit p40 is fused to the C-terminus of p35 through a non-cleavable peptide linker. The second masking moiety is fused to the C-terminus of the other Fc domain polypeptide through a cleavable linker and the first masking moiety is fused to the C-terminus of the second masking moiety through a cleavable peptide linker. FIG. 1B shows the IL-12 subunit p40 fused to the C-terminus of one of the Fc domain polypeptides, while the IL-12 subunit p35 is fused to the C-terminus of p40 through a non-cleavable peptide linker. The first masking moiety is fused to the C-terminus of the other Fc domain polypeptide through a cleavable linker and the second masking moiety is fused to the C-terminus of the first masking moiety through cleavable peptide linker.

FIGS. 2A and 2B show an illustration of the structure of an IL-12 prodrug comprising an IL-12 p40 subunit (p40), an IL-12 p35 subunit (p35), a first masking moiety (MM1; white box), a second masking moiety (MM2; black box), a carrier (C), and one cleavable peptide linker. FIG. 2A shows the IL-12 subunit p35 fused to the C-terminus of one of the Fc domain polypeptides, while the IL-12 subunit p40 is fused to the C-terminus of p35 through a non-cleavable peptide linker. The second masking moiety is fused to the C-terminus of the other Fc domain polypeptide through a non-cleavable linker and the first masking moiety is fused to the C-terminus of the second masking moiety through cleavable peptide linker. FIG. 2B shows the IL-12 subunit p40 fused to the C-terminus of one of the Fc domain polypeptides, while the IL-12 subunit p35 is fused to the C-terminus of p40 through a non-cleavable peptide linker. The first masking moiety is fused to the C-terminus of the other Fe domain polypeptide through a non-cleavable linker and said second masking moiety is fused to the C-terminus of the first masking moiety through cleavable peptide linker.

FIGS. 3A and 3B show an illustration of the structure of an IL-12 prodrug comprising an IL-12 p40 subunit (p40), an IL-12 p35 subunit (p35), a first masking moiety (MM1; white box), a second masking moiety (MM2; black box), a carrier (C), and two cleavable peptide linkers. FIG. 3A shows an IL-12 subunit p35 fused to the C-terminus of one of the Fc domain polypeptides, while an IL-12 subunit p40 is fused to the C-terminus of the other Fc domain polypeptide, both through a non-cleavable peptide linker. The second masking moiety is fused to the C-terminus of p35 through a cleavable linker, while the first masking moiety is fused to the C-terminus of p40 through cleavable peptide linker. FIG. 3B shows an IL-12 subunit p35 fused to the C-terminus of one of the Fc domain polypeptides, while the IL-12 subunit p40 is fused to the C-terminus of the other Fc domain polypeptide, both through a non-cleavable peptide linker. The second masking moiety is fused to the C-terminus of p40 through a cleavable linker, while the first masking moiety is fused to the C-terminus of p35 through cleavable peptide linker.

FIGS. 4A and 4B show an illustration of the structure of an IL-12 prodrug comprising an IL-12 p40 subunit (p40), an IL-12 p35 subunit (p35), a first masking moiety (MM1; white box), a second masking moiety (MM2; black box), a carrier (C), and one cleavable peptide linker. FIG. 4A shows the IL-12 subunit p35 fused to the C-terminus of one of the Fc domain polypeptides, while the IL-12 subunit p40 is fused to the C-terminus of the other Fc domain polypeptide, both through a non-cleavable peptide linker. The second masking moiety is fused to the C-terminus of p35 through a cleavable linker and the first masking moiety is fused to the C-terminus of p40 through non-cleavable peptide linker. FIG. 4B shows an IL-12 subunit p35 fused to the C-terminus of one of the Fc domain polypeptides, while the IL-12 subunit p40 is fused to the C-terminus of the other Fc domain polypeptide, both through a non-cleavable peptide linker. The second masking moiety is fused to the C-terminus of p40 through a cleavable linker and the first masking moiety is fused to the C-terminus of p35 through non-cleavable peptide linker.

FIGS. 5A and 5B show an illustration of the structure of an IL-12 prodrug comprising an IL-12 p40 subunit (p40), an IL-12 p35 subunit (p35), a masking moiety (MM), and one cleavable peptide linker. FIG. 5A shows the IL-12 subunit p35 fused to the C-terminus of one of the Fe domain polypeptides, while the IL-12 subunit p40 is fused to the C-terminus of p35, both through a non-cleavable peptide linker. In addition, the masking moiety is fused to the C-terminus of the second Fe through a cleavable linker. FIG. 5B shows the IL-12 subunit p40 fused to the C-terminus of one of the Fe domain polypeptides, while the IL-12 subunit p35 is fused to the C-terminus of p40, both through a non-cleavable peptide linker. In addition, the masking moiety is fused to the C-terminus of the second Fe through a cleavable linker.

FIGS. 6A and 6B show an illustration of the structure of an IL-12 prodrug comprising an IL-12 p40 subunit (p40), an IL-12 p35 subunit (p35), a masking moiety (MM), a carrier (C), and no cleavable peptide linker. FIG. 6A shows the IL-12 subunit p35 fused to the C-terminus of one of the Fe domain polypeptides, while the IL-12 subunit p40 is fused to the C-terminus of the other Fe domain polypeptide, both through a non-cleavable peptide linker. In addition, the masking moiety is fused to the C-terminus of p35 through a non-cleavable linker. FIG. 6B shows the IL-12 subunit p40 fused to the C-terminus of one of the Fe domain polypeptides, while the IL-12 subunit p35 is fused to the C-terminus of p40, both through a non-cleavable peptide linker. In addition, said masking moiety is fused to the C-terminus of the second Fe through a non-cleavable linker.

FIGS. 7A and 7B show an illustration of the structure of an IL-12 prodrug comprises three polypeptide chains, which comprises an IL-12 p40 subunit (p40), an IL-12 p35 subunit (p35), a masking moiety (MM), and a carrier (C). FIG. 7A shows the IL-12 subunit p35 fused to the C-terminus of one of the Fe domain polypeptides, the masking moiety is fused to the C-terminus of the other Fe domain polypeptide, both optionally through a non-cleavable linker. The p40 is the third polypeptide chain, which is linked to the p35 subunit through a disulfide bond. FIG. 7B shows the IL-12 subunit p35 fused to the C-terminus of one of the Fe domain polypeptides optionally through a non-cleavable peptide linker, the masking moiety is fused to the C-terminus of the other Fe domain polypeptide through a cleavable peptide linker. The p40 is the third polypeptide chain, which is linked to the p35 subunit through a disulfide bond.

FIGS. 8A and 8B show an SDS-PAGE analysis. FIG. 8A shows the SDS-PAGE analysis of the Protein A Affinity chromatography pool samples of the IL-12 prodrugs JR3.105.2, JR3.105.3, JR3.105.4, and JR3.105.5 with structures as illustrated in FIG. 5B and sequences as shown in Table 2. The samples were run under reduced and non-reduced conditions. The samples were of reasonable purity though some levels of the activated versions of the prodrugs are visible. FIG. 8B shows the SDS-PAGE analysis of the prodrug JR3.105.5 sample prior to and after protease treatment in order to activate the prodrug. The data showed that the majority of the prodrug sample were activated.

FIG. 9 shows a cell-based activity analysis of the IL-12 prodrug samples prior to and after protease treatment. The prodrugs treated with protease are labeled as JR3.105.2-Clv, JR3.105.3-Clv, JR3.105.4-Clv, and JR3.105.5-Clv. Significant activation was observed with the Prodrug JR3.105.5 after the treatment with protease.

FIG. 10 shows the EC₅₀ values of IL-12 prodrug samples tested in the cell-based assay, as shown in FIG. 9. The EC₅₀ value for the Prodrug JR3.105.5 was approximately 0.9 nM prior to activation and approximately 0.05 nM after activation.

### DETAILED DESCRIPTION OF THE INVENTION

As used herein and in the appended claims, the singular forms “a,” “or,” and “the” include plural referents unless the context clearly dictates otherwise. Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The terms “cytokine agonist polypeptide” or “cytokine moiety” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine mask” or “masking moiety” refers to a moiety (e.g., a polypeptide) that binds to a cytokine, thereby inhibiting the cytokine from binding to its receptor on the surface of a target cell and/or exerting its biological functions while being bound by the mask. Examples of a cytokine mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### I. IL-12 Prodrugs

The present disclosure provides IL-12 prodrugs that are metabolized in vivo to become active IL-12 therapeutics. The IL-12 prodrugs have fewer side effects, better in vivo PK profiles (e.g., longer half-life) and better target specificity, and are more efficacious as compared to prior IL-12 therapeutics. The present IL-12 prodrugs comprise an IL-12 agonist polypeptide, at least one masking moiety (M), a carrier (C), and one or more cleavable or non-cleavable peptide linkers that link the masking moiety to the agonist polypeptide or the carrier. In some embodiments, the IL-12 prodrugs comprise an IL-12 agonist polypeptide, a first masking moiety (MM1), a second masking moiety (MM2), a carrier (C), and one or more cleavable or non-cleavable peptide linkers that link the masking moieties to the agonist polypeptide or the carrier.

IL-12 comprises subunits p40 and p35. Subunit p40 comprises an amino acid as shown in SEQ ID NO: 5 and p35 comprises an amino acid sequence as shown in SEQ ID NO: 6. In some embodiments, an IL-12 analog comprises subunit p40 with an amino acid that is at least 90% identical to SEQ ID NO: 5. In other embodiments, an IL-12 analog comprises subunit p35 with an amino acid sequence that is at least 90% identical to SEQ ID NO: 6.

In some embodiments, the IL-12 prodrugs comprise at least one masking moiety. The masking moieties may be linked to the cytokine moiety or to the carrier moiety through a peptide linker. In some embodiments, the peptide linker is a cleavable peptide linker. In some embodiments, the cleavable peptide linker comprises one or more cleavable moieties, which are substrates of proteases typically found at a tumor site. The mask inhibits the cytokine moiety's biological functions while the mask is binding to it. In specific embodiments, the mask inhibits a biological activity of IL-12 or its analog. The prodrugs may be activated at a target site (e.g., at a tumor site or the surrounding environment) in the patient by cleavage of the linker and the consequent release of the cytokine mask from the prodrug, exposing the previously masked cytokine moiety and allowing the cytokine moiety to bind to its receptor on a target cell and exert its biological functions on the target cell.

In some embodiments, the carriers for the IL-12 prodrugs are antigen-binding moieties that bind an antigen at a target site (e.g., tumor surface). In some embodiments, the present IL-12 prodrugs are metabolized to become active in the body at a target site targeted by the carrier. In further embodiments, the carrier in the prodrug is an antibody targeting a tumor antigen such that the IL-12 prodrug is delivered to a tumor site in a patient and is metabolized locally (e.g., inside or in the vicinity of the tumor microenvironment) through cleavage of a linker linking the cytokine mask to the carrier or the cytokine moiety, making the cytokine moiety available to interact with its receptor on a target cell and stimulating the target immune cells locally.

In some embodiments, the carrier is selected from an albumin, a Fc fragment, a polyethylene glycol (PEG), or an antibody or antigen-binding fragment thereof.

A. Masking Moieties of the IL-12 Prodrugs

In some embodiments, the masking moiety (M, MM1, or MM2) for IL-12 from an IL-12 receptor beta1 subunit extracellular domain or a fragment thereof, an IL-12 receptor beta2 subunit extracellular domain or a fragment thereof, and a scFv or Fab with specificity to p40 or p35. In some embodiments, the masking moiety inhibits a biological activity of IL-12 or its analog. In some embodiments, the masking moiety comprises a scFv, wherein the scFv has an amino acid sequence at least 99% identical to SEQ ID NOs: 7-11 and 12. In some embodiments, the masking moiety is an IL-12 receptor extracellular domain (ECD) or its functional analog, wherein the IL-12 receptor is IL-12 receptor beta1. In some embodiments, the masking moiety is IL-12 receptor extracellular domain (ECD), its functional analog, or a fragment thereof, wherein the IL-12 receptor is IL-12 receptor beta1. In some embodiments, IL-12 receptor beta1 has an amino acid sequence that is at least 95% identical to SEQ ID NO: 137. In some embodiments, the prodrug comprises two masking moieties MM1 and MM2; wherein at least one of the masking moieties comprises a scFv or Fab, which binds to p40 or p35, and reduces or inhibits the binding of IL-12 to IL-12Rβ1 or IL-12Rβ2. In some embodiments, the scFv or Fab comprises the same light chain CDRs and heavy chain CDRs as IL-12 antibody briakinumab or ustekinumab. In some embodiments, the scFv or Fab comprises the same light chain CDRs and heavy chain CDRs as an IL-12 antibody selected from PMA204, Antibody 1, Antibody 50, Antibody 68, Antibody 80, and Antibody 136 (see U.S. Pat. No. 8,563,697).

In some embodiments, both masking moieties (MM1 and MM2) are scFvs or Fabs; wherein the first masking moiety binds to p40 and reduces or inhibits the binding of IL-12 to IL-12Rβ1, and the second masking moiety binds to p40 or p35, and reduces or inhibits the binding of IL-12 to IL-12Rβ2. In some embodiments, the first masking moiety comprises the same light chain CDRs and heavy chain CDRs as an IL-12 antibody briakinumab or ustekinumab. In some embodiments, the second masking moiety comprises the same light chain CDRs and heavy chain CDRs as an IL-12 antibody selected from PMA204, Antibody 1, Antibody 50, Antibody 68, Antibody 80, and Antibody 136 (see U.S. Pat. No. 8,563,697).

B. Carrier Moieties of the Prodrugs

The carrier moieties of the present prodrugs may be an antigen-binding moiety, or a moiety that is not antigen-binding. The carrier moiety may improve the PK profiles, such as serum half-life, of the cytokine agonist polypeptide and may also target the cytokine agonist polypeptide to a target site in the body, such as a tumor site.

1. Non-Antigen-Binding Carrier Moieties

Non-antigen-binding carrier moieties may be used for the present prodrugs. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

The carrier moiety of the prodrug may comprise an albumin (e.g., human serum albumin) or a fragment thereof. In some embodiments, the albumin or albumin fragment is about 85% or more, about 90% or more, about 91% or more, about 92% or more, about 93% or more, about 94% or more, about 95% or more, about 96% or more, about 97% or more, about 98% or more, about 99% or more, about 99.5% or more, or about 99.8% or more identical to human serum albumin or a fragment thereof.

In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment

In some embodiments, the carrier is an antibody Fc fragment. Fc is a dimeric molecule that has two N-terminals and two C-terminals. In some embodiments, the cytokine moiety can be fused to one Fc polypeptide in a dimeric Fc fragment, and the masking moieties can be fused to the 2′ Fc polypeptide. In a preferred embodiment, both the cytokine moiety and the masking moiety are fused to the C-terminal of each polypeptide chain of the dimeric Fc fragment. In some embodiments, both the cytokine moiety and the masking moieties are fused to the N-terminal of each polypeptide chain of the dimeric Fc fragment. In either case, at least one of the masking moieties is fused to the Fc polypeptide directly or indirectly through a cleavable peptide linker.

In some embodiments, the carrier moiety is an Fc domain comprising a first and a second polypeptide chain (i.e., two different heavy chains), wherein the polypeptide chains of the prodrug comprise molecular formulae (from N-terminus to C-terminus) selected from one of the following pairs:

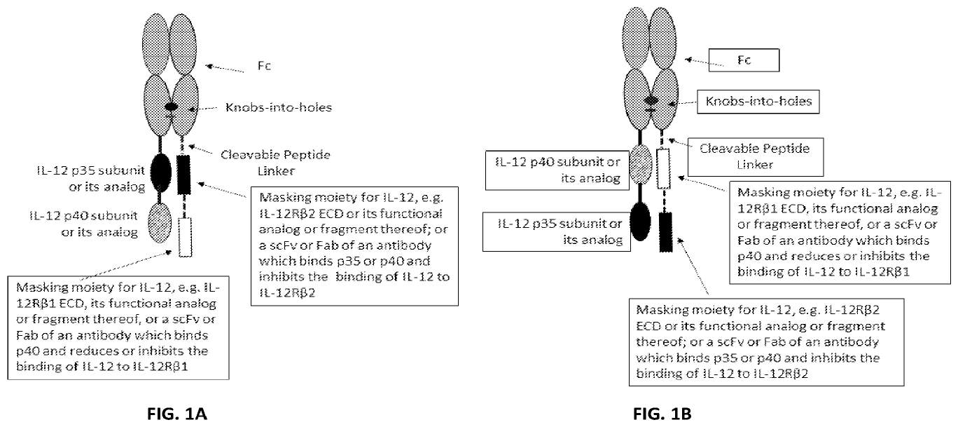

a. F1-PL1-A-PL2-B, F2-CL1-MM2-CL2-MM1 (FIG. 1A); and

b. F1-PL1-B-PL2-A, F2-CL1-MM1-CL2-MM2 (FIG. 1B);

wherein F1 and F2 are subunits of the carrier moiety (e.g., Fe domain), which form a heterodimer; PL1 and PL2 are peptide linkers; CL1 and CL2 are cleavable peptide linkers; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In some embodiments, the carrier moiety is an Fc domain comprising a first and a second polypeptide chain (i.e., two different heavy chains), wherein the polypeptide chains of the prodrug comprise molecular formulae (from N-terminus to C-terminus) selected from one of the following pairs:

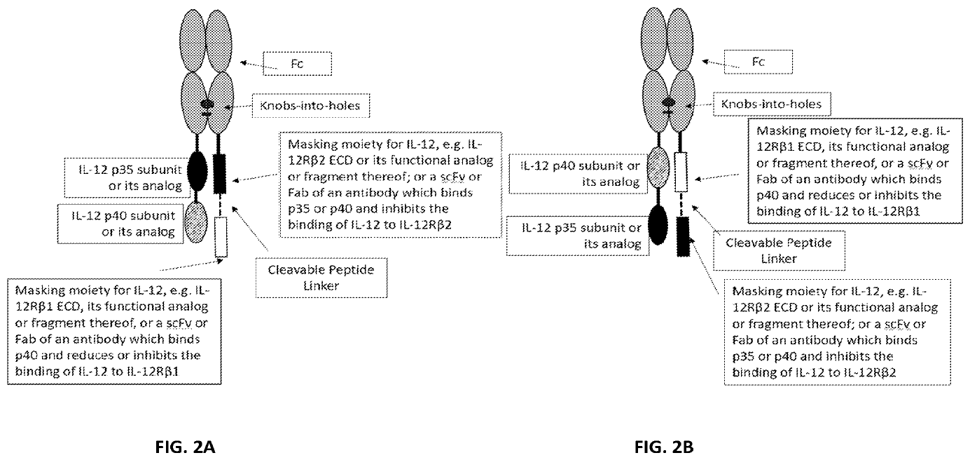

a. F1-PL1-A-PL2-B, F2-PL3-MM2-CL-MM1 (FIG. 2A); and

b. F1-PL1-B-PL2-A, F2-PL3-MM1-CL-MM2 (FIG. 2B);

wherein F1 and F2 are subunits of the carrier moiety (e.g., Fc domain), which form a heterodimer; PL1, PL2, and PL3 are peptide linkers; CL is a cleavable peptide linker; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In some embodiments, the carrier moiety is an Fc domain comprising a first and a second polypeptide chain (i.e., two different heavy chains), wherein the polypeptide chains of the prodrug comprise molecular formulae (from N-terminus to C-terminus) selected from one of the following pairs:

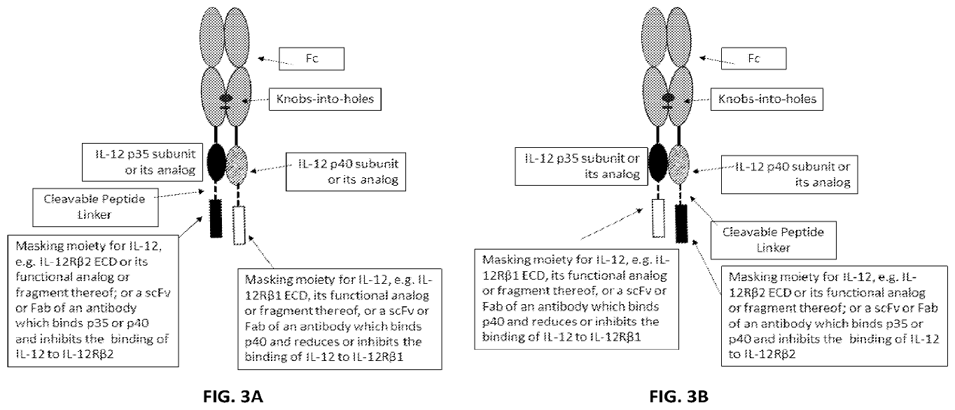

a. F1-PL1-A-CL1-MM2, F2-PL2-B-CL2-MM1 (FIG. 3A); and

b. F1-PL1-A-CL1-MM1, F2-PL2-B-CL2-MM2 (FIG. 3B);

wherein F1 and F2 are subunits of the carrier moiety (e.g., Fc domain), which form a heterodimer; PL1 and PL2 are peptide linkers; CL1 and CL2 are cleavable peptide linkers; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In some embodiments, the carrier moiety is an Fe domain comprising a first and a second polypeptide chain (i.e., two different heavy chains), wherein the polypeptide chains of the prodrug comprise molecular formulae (from N-terminus to C-terminus) selected from one of the following pairs:

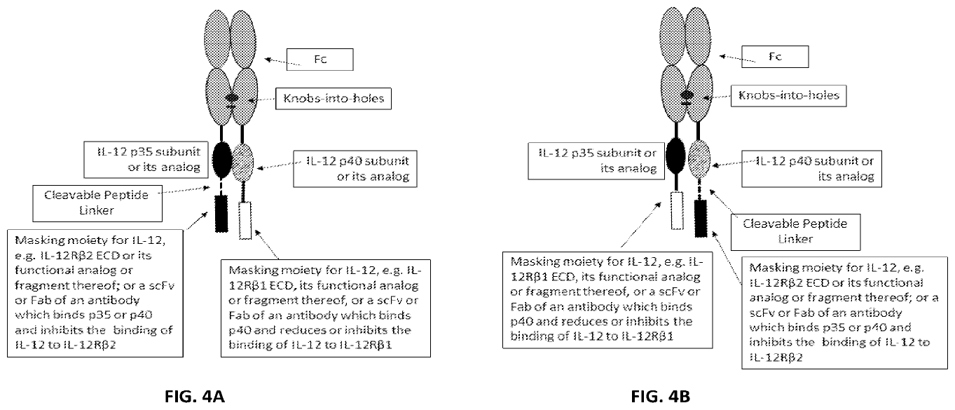

a. F1-PL1-A-CL-MM2, F2-PL2-B-PL3-MM1 (FIG. 4A); and

b. F1-PL1-A-PL2-MM1, F2-PL3-B-CL-MM2 (FIG. 4B);

wherein F1 and F2 are subunits of the carrier moiety (e.g., Fc domain), which form a heterodimer; PL1, PL2, and PL3 are peptide linkers; CL is a cleavable peptide linker; MM1 is the first masking moiety, which binds to p40 and reduces or inhibits the binding of IL-12 to IL-12Rβ1; MM2 is the second masking moiety, which binds to p35 and/or p40 and reduces or inhibits the binding of IL-12 to IL-12Rβ2; A is an IL-12 p35 subunit; and B is an IL-12 p40 subunit.

In some embodiments, the carrier moiety is an Fc domain comprising a first and a second polypeptide chain (i.e., two different heavy chains), wherein the polypeptide chains of the prodrug comprise molecular formulae (from N-terminus to C-terminus) selected from one of the following pairs:

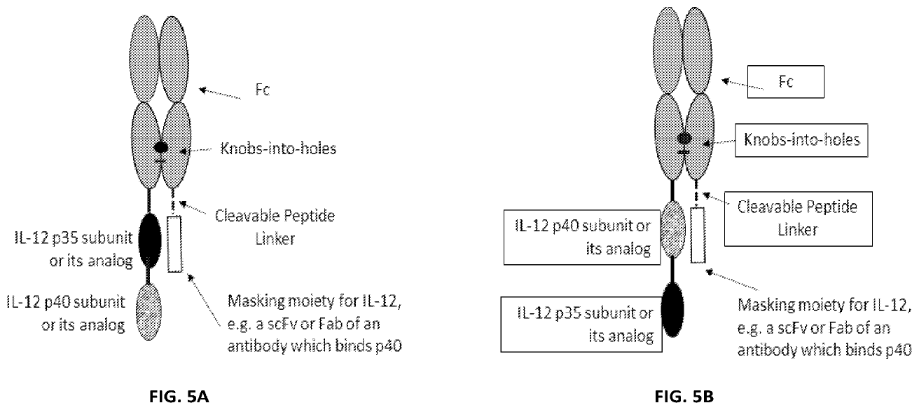

a. F1-PL1-A-PL2-B, F2-CL-M (FIG. 5A); and

b. F1-PL1-B-PL2-A, F2-CL-M (FIG. 5B);

wherein F1 and F2 are subunits of the carrier moiety (e.g., Fc domain), which form a heterodimer; PL1 and PL2 are peptide linkers; CL is a cleavable peptide linker; M is a masking moiety that binds to p40 and reduces or inhibits the binding of IL-12 to IL-12Rβ1; A is an IL-12 p35 subunit; and B is an IL-12 p40 subunit.

In some embodiments, the carrier moiety is an Fc domain comprising a first and a second polypeptide chain (i.e., two different heavy chains), wherein the polypeptide chains of the prodrug comprise molecular formulae (from N-terminus to C-terminus) selected from one of the following pairs:

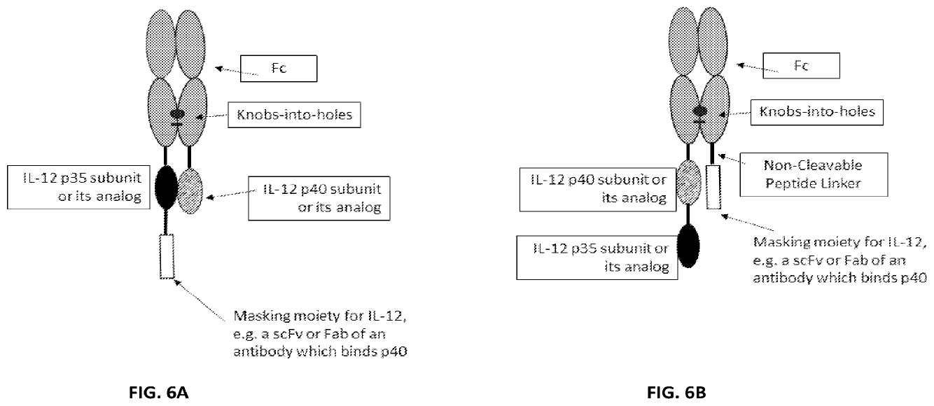

a. F1-PL1-A-PL2-M, F2-PL3-B (FIG. 6A); and

b. F1-PL1-B-PL2-A, F2-PL3-M (FIG. 6B);

wherein F1 and F2 are subunits of the carrier moiety (e.g., Fc domain), which form a heterodimer; PL1, PL2, and PL3 are peptide linkers; M is a masking moiety that binds to p40 and reduces or inhibits the binding of IL-12 to IL-12Rβ1; A is an IL-12 p35 subunit; and B is an IL-12 p40 subunit.

In some embodiments, the carrier moiety is an Fc domain comprising a first and a second polypeptide chain (i.e., two different heavy chains), wherein the polypeptide chains of the prodrug comprise molecular formulae (from N-terminus to C-terminus) selected from one of the following pairs:

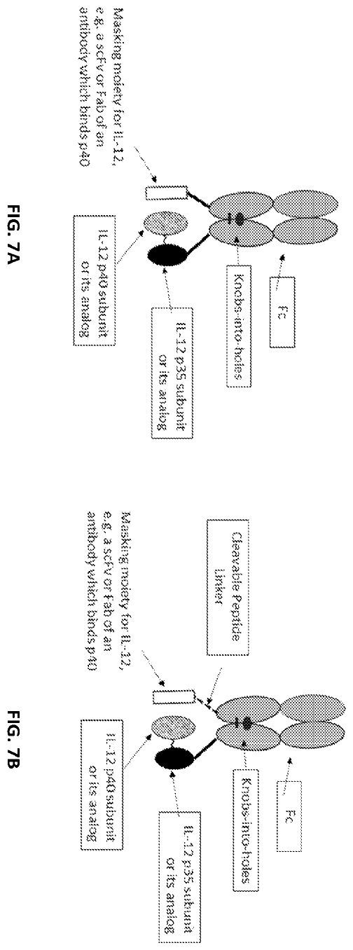

a. F1-PL1-M, F2-PL2-A-SS-B (FIG. 7A); and

b. F1-CL-M, F2-PL1-A-SS-B (FIG. 7B);

wherein F1 and F2 are subunits of the carrier moiety (e.g., Fc domain), which form a heterodimer; PL1 and PL2 are peptide linkers; M is a masking moiety that binds to p40 and reduces or inhibits the binding of IL-12 to IL-12Rβ1; CL is a cleavable peptide linker; A is an IL-12 p35 subunit; and B is an IL-12 p40 subunit; and SS is a disulfide bond.

2. Antigen-Binding Carrier Moieties

The carrier moiety may be an antibody or an antigen-binding fragment thereof, or an immunoadhesin. In some embodiments, the antigen-binding moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide. In some embodiments, the antigen-binding moiety comprises a full-length antibody heavy chain or a full-length antibody light chain. In some embodiments, the antigen-binding moiety includes an antibody heavy chain fragment or an antibody light chain fragment.

In some embodiments, the cytokine moiety is fused to the C-terminus of one of the heavy chains of an antibody, and the cytokine's mask is fused to the C-terminus of the other heavy chain of the antibody through a peptide linker (optionally a cleavable linker), wherein the two heavy chains optionally contain mutations that allow the specific pairing of the two different heavy chains.

Strategies of forming heterodimers for Fc-fusion polypeptides or bispecific antibodies are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech. (1998) 16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the prodrug comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG₁ (see Dall'Acqua et al., J Biol Chem. (2006) 281: 23514-24; and Robbie et al., Antimicrob Agents Chemother. (2013) 57(12):6147-53). In further embodiments, the YTE mutations are present in the antibody moiety in addition to the knobs-into-holes mutations. In particular embodiments, the antibody moiety has YTE, LALA and knobs-into-holes mutations or any combination thereof.

In some embodiments, the antibody binds to PD-L1. In some embodiment, the antibody binds to CEA. In some embodiment, the antibody binds to an antigen on a tumor cell, for examples, 5T4, FAP, Trop-2, PD-L1, HER-2, EGFR, Claudin 18.2, DLL-3, GCP3, and CEA. The antibody may or may not have Antibody-Dependent Cellular Cytotoxicity (ADCC) activity. The antibody may also be further conjugated with cytotoxic drugs. In some embodiment, the antibody binds to a target on the surface of an immune cell and has the ability to activate said immune cell and enhance its anti-cancer activity, for examples, PD-1 antibody, LAG3 antibody, TIGIT antibody, TGF-beta antibody, and CTLA4 antibody.

The antigen-binding moiety can bind an antigen on the surface of a cell, such as a cancer cell. In some embodiments, the antigen-binding moiety is a bispecific antigen-binging moiety, can bind to two different antigens or two different epitopes on the same antigen. In some embodiments, the antigen-binding moiety binds to Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1) 1, vascular endothelial growth factor receptor 2 (VEGFR2), Nectin-4, Liv-1, glycoprotein NMB (GPNMB), prostate-specific membrane antigen (PSMA), Trop-2, carbonic anhydrase IX (CA9), endothelin B receptor (ETBR), Thomsen-Friedenrech antigen (TF), sodium-dependent phosphate transport protein 2B (NaPi2b), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, programmed death ligand 1 (PD-L1), Claudin 18.2, Claudin 6, PSMA, or FAP-alpha. In some embodiments, the antigen-binding moiety binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moiety binds to an epitope located after the 374^{th} amino acid of GPC3. In some embodiments, the antigen-binding moiety binds to a heparin sulfate glycan of GPC3. In some embodiments, the antigen-binding moiety binds to Claudin 18.2 and does not bind to Claudin 18.1. In some embodiments, the antigen-binding moiety binds to Claudin 18.1 with at least 10 times weaker binding affinity than to Claudin 18.2.

The antigen-binding moiety can bind an antigen on the surface of a cell, such as an immune cell, for example T cells, NK cells, and macrophages. In some embodiments, the antigen-binding moiety is a bispecific antigen-binging moiety, can bind to two different antigens or two different epitopes on the same antigen. In some embodiments, the antigen-binding moiety binds to PD-1, LAG-3, TIM-3, CTLA-4, or TGF-beta.

In some embodiments, the antigen-binding moiety (carrier moiety) includes an antibody or fragment thereof known in the art that binds to PD-1 and disrupts the interaction between the PD-1 and its ligand (PD-L1) to stimulate an anti-tumor immune response. In some embodiments, the antibody or antigen-binding portion thereof binds specifically to PD-1. For example, antibodies that target PD-1 and which can find use in the present invention include, but are not limited to, nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)-BioXcell cat # BP0146. Other suitable anti-PD-1 antibodies include those disclosed in U.S. Pat. No. 8,008,449. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in combination treatment methods disclosed herein. As an example, antibodies that target PD-L1 include BMS-936559 (Bristol-Myers Squibb) and MPDL3280A (Genetech; currently in human trials). Other suitable antibodies that target PD-L1 are disclosed in U.S. Pat. No. 7,943,743. It will be understood by one of ordinary skill that any antibody which binds to PD-1 or PD-L1, disrupts the PD-1/PD-L1 interaction, and stimulates an anti-tumor immune response, is suitable for use in the combination treatment methods disclosed herein.

In some embodiments, wherein the carrier is an antibody against human PD-L1 is selected from ASKB1296, avelumab, atezolizumab and durvalumab.

Exemplary antigen-binding moieties include trastuzumab, rituximab, brentuximab, cetuximab, panitumumab, GC33 (or a humanized version thereof), anti-EGFR antibody mAb806 (or a humanized version thereof), anti-FAP-alpha antibody sibrotuzumab (BIBH1), and fragments thereof. In some embodiments, the antigen-binding moiety that has at least 90%, 91%, 92%, 93%, 94%, 95%, 96%, 97%, 98%, or 99% identity to trastuzumab, rituximab, brentuximab, cetuximab, or panitumumab, GC33 (or a humanized version thereof), anti-EGFR antibody mAb806 (or a humanized version thereof), sibrotuzumab (BIBH1), or a fragment thereof. In some embodiments, the antigen-binding moiety has an antibody heavy chain with at least 90%, 91%, 92%, 93%, 94%, 95%, 96%, 97%, 98%, or 99% identity to the antibody heavy chain of trastuzumab, rituximab, brentuximab, cetuximab, panitumumab, GC33 (or a humanized version thereof), anti-EGFR antibody mAb806 (or a humanized version thereof), sibrotuzumab (BIBH1), or a fragment thereof. In some embodiments, the antigen-binding moiety has an antibody light chain with at least 90%, 91%, 92%, 93%, 94%, 95%, 96%, 97%, 98%, or 99% identity to the antibody light chain of trastuzumab, rituximab, brentuximab, cetuximab, panitumumab, GC33 (or a humanized version thereof), anti-EGFR antibody mAb806 (or a humanized version thereof), sibrotuzumab (BIBH1), or a fragment thereof.

In some embodiments, the antigen-binding moiety comprises the six complementarity-determining regions (CDRs) of trastuzumab, rituximab, brentuximab, cetuximab, panitumumab, GC33, anti-EGFR antibody mAb806, or sibrotuzumab (BIBH1). A number of CDR delineations are known in the art and are encompassed herein. A person of skill in the art can readily determine a CDR for a given delineation based on the sequence of the heavy or light chain variable region. The “Kabat” Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, J. Mol. Biol. (1987) 196:901-917). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid number of antibodies refers to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat | | | | |
| Numbering) | | | | |
| VH-CDR1 | H31-H35 | H26-H35 | H26-H32 | H30-H35 |
| (Chothia | | | | |
| Numbering) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the IL-12 prodrug of the present disclosure comprises a carrier comprising an antigen-binding moiety; wherein the antigen-binding moiety binds to Trop-2. In some embodiments, the IL-12 prodrug is used to treat patients with solid tumors. In some embodiments, the prodrug is used to treat triple negative breath cancer, urothelial cancer, small-cell lung cancer, pancreatic cancer, hilar cholangiocarcinoma, cervical cancer, and gastric cancer.

In some embodiment, the IL-12 prodrug comprises a carrier comprising an antigen-binding moiety; wherein the antigen-binding moiety binds to 5T4. In some embodiments, the IL-12 prodrug is used to treat patients with solid tumor. In some embodiments, the prodrug is used to treat triple negative breath cancer, small-cell lung cancer, non-small cell lung cancer, pancreatic cancer, ovarian cancer, and gastric cancer.

In some embodiments, the IL-12 prodrug comprises a carrier comprising an antigen-binding moiety; wherein said antigen-binding moiety binds to Claudin 18.2. In some embodiments, the IL-12 prodrug is used to treat patients with pancreatic cancer and gastric cancer.

In some embodiments, the IL-12 prodrug comprises a carrier comprising an antigen-binding moiety; wherein said antigen-binding moiety binds to EGFR Type III. In some embodiment, said IL-12 prodrug is used to treat patients with glioblastoma and colon cancer.

In some embodiment, the IL-12 prodrugs are used in combination with an immune checkpoint blockade, such as a PD-1 antibody or an PD-1 antibody fragment thereof.

C. Linker Components of the Prodrugs

The IL-12 agonist polypeptide may be fused to the carrier moiety with or without a peptide linker. The peptide linker may be noncleavable. In some embodiments, the peptide linker is selected from SEQ ID NOs: 29-33.

The IL-12 mask may be fused to the cytokine moiety, to the carrier, or to another mask through a noncleavable linker or a cleavable linker. The cleavable linker may contain one or more (e.g., two or three) cleavable moieties (CM). Each CM may be a substrate for an enzyme or protease selected from legumain, plasmin, TMPRSS-3/4, MMP2, MMP9, MT1-MMP, cathepsin, caspase, human neutrophil elastase, beta-secretase, uPA, and PSA. Examples of cleavable linkers include, without limitation, those comprising an amino acid sequence selected from SEQ ID NOs: 34-54.

D. IL-12 Prodrugs with an Additional Effector Polypeptide

1. IL-2 Agonist Polypeptides

In some embodiments, the IL-12 prodrug of the present disclosure further comprises another cytokine or effector moiety. In some embodiments, the second cytokine moiety comprises an IL-2 agonist polypeptide.

In some embodiments, the IL-2 agonist polypeptide in the prodrug comprises an amino acid sequence at least 85%, at least 90%, at least 95%, or 100% identical to SEQ ID NO:1. In some embodiments, the IL-2 agonist polypeptide contains mutations which lead to the significantly reduced affinity to the high-affinity trimeric IL-2 receptor. The high-affinity trimeric IL-2 receptor comprises alpha, beta, and gamma IL-2 receptor subunits. Optionally, the IL-2 agonist polypeptide further comprises mutations that lead to significantly reduced binding affinity to the moderate-affinity IL-2 receptor, which comprises the IL-2 receptor beta and gamma subunits. In some embodiments, the IL-2 agonist polypeptide comprises one or more mutations at residues R38, F42, F44, Y45, E62, E68, L72, and A73 according to the numbering of the human IL-2 with amino acid sequence of SEQ ID NO: 1. In some embodiments, the IL-2 agonist polypeptide further comprises one or more mutations at residues D20, N88, N90, and Q126 according to the numbering of the human IL-2 with amino acid sequence of SEQ ID NO: 1. Additional mutations at residues T3 and/or C125 are optionally included.

In some embodiments, the IL-2 agonist polypeptide of the IL-2 prodrug comprises mutations at residues F42N/F44T, A73T, and/or N90T; wherein the mutations are referred to according to the numbering of the human IL-2 with amino acid sequence of SEQ ID NO: 1.

In some embodiments, the IL-2 agonist polypeptide of the IL-2 prodrug comprises mutations selected from one of the following combinations:

T3A, R38A, F42I, Y45N, E62L, E68V, C125S;

T3A, R38K, F42K, Y45R, E62L, E68V, C125S;

T3A, R38A, F42A, Y45A, E62A, C125S;

T3A, R38S, F42A, Y45A, E62A, C125S; or

R38S, F42A, Y45A, E62A; and

wherein said mutations are referred to according to the numbering of the human IL-2 with amino acid sequence of SEQ ID NO: 1.

In some embodiments, the IL-2 agonist polypeptide is at least 30 times lower affinity in binding to the high-affinity trimeric IL-2 receptor comprising the alpha, beta, and gamma subunits as compared to the wild type IL-2; wherein the polypeptide comprises one or more mutations at R38, F42, Y45, E62, E68, and L72; wherein the mutations are referred to according to the numbering of the human IL-2 with amino acid sequence of SEQ ID NO: 1. In some embodiments, the agonist polypeptide of IL-2 further comprises one or more mutations at residues A3, D20, N88, C125 and/or Q126 according to the numbering of the human IL-2 with amino acid sequence of SEQ ID NO: 1.

In some embodiments, the IL-2 agonist peptide comprises an amino acid sequence selected from SEQ ID NOs: 74 and 75.

2. IL-15 Agonist Polypeptides

In some embodiments, the IL-12 prodrug of the present disclosure further comprises another cytokine or effector moiety. In some embodiments, the second cytokine moiety comprises an IL-15 agonist polypeptide. In some embodiments, the IL-15 agonist polypeptide may optionally comprise the Sushi domain of the IL-15 receptor alpha subunit.

In some embodiments, the IL-15 moiety is an IL-15 mutein comprising at least 1, 2, 3, 4, or 5 mutations at positions selected from N1, N4, I6, S7, D8, K10, K11, E46, D61, T62, E64, N65, I68, L69, N72, V63, L66, I67, A70, N71, Q108, N112 of human IL-15. Exemplary IL-15 muteins are those with one or more mutations selected from N1A, N1D, N4A, N4D, I6T, S7A, D8A, DAT, D8E, D8N, K10A, K10D, K11A, K11D, D61A, D61N, T62L, T62A, E64A, E64L, E64K, E64Q, N65A, N65L, N65D, L66D, L66E, I67D, I67E, I68S, 168E, L69S, L69E, N72A, N72D, V63E, V63D, L66E, L66D, I67E, I67D, Q108E, and N112A. In some embodiments, the IL-15 moiety comprises a mutation at positions selected from E46, V49, L45, S51, and L52. Unless otherwise indicated, all residue numbers in IL-15 and IL-15 muteins described herein are in accordance with the numbering in SEQ ID NO: 4. In other embodiments, the IL-15 moiety comprises an amino acid sequence that is at least 90%, 91%, 92%, 93%, 94%, 95%, 96%, 97%, 98%, or 99% identical to SEQ ID NO: 4.

In particular embodiments, the IL-15 mutein contains mutations selected from N1D/D61N, N1D/E64Q, N4D/D61N, N4D/E64Q, D8N/D61N, D8N/E64Q, D30N/E64Q/N65D, D61N/E64Q, E64Q/Q108E, N1D/N4D/D8N, D61N/E64Q/N65D, N1D/D61N/E64Q, N1D/D61N/E64Q/Q108E, and N4D/D61N/E64Q/Q108E.

In some embodiments, the present IL-15 prodrug comprises an IL-15Ra Sushi domain. The Sushi domain may be fused to the carrier directly or to the IL-15 cytokine moiety, optionally through a linker (e.g., a noncleavable or cleavable peptide linker). The masking moiety may be fused to the Sushi domain or to the carrier through a cleavable or noncleavable peptide linker. In a particular embodiment, the Sushi domain is fused to the carrier and the cytokine moiety is fused to the Sushi domain through a peptide linker.

3. IL-2/IL-15 Masking Moieties

In some embodiments, masking moieties of the additional cytokine moieties disclosed herein comprise a peptide, monomer, or dimer of a soluble extracellular domain of an IL-2 receptor beta subunit, a soluble extracellular domain of IL-2 receptor beta and gamma subunit fusion protein or complex, or antibodies that bind to IL-2 or IL-15 and interfere with the binding of IL-2, IL-2 muteins, or IL-15 to the dimeric IL-2 receptor. In some embodiments, an IL-2 masking moiety also inhibits or reduces the biological activities of IL-2 or IL-15. In some embodiments, the IL-2 masking moiety is a peptide identified from a peptide library through screening. In some embodiments, the IL-2 masking moiety is an antibody or fragment thereof that blocks the binding of IL-2 or IL-2 mutants to an IL-2 receptor. In some embodiments, the IL-2 masking moiety is an scFv, a Fab or a single chain Fab having the same CDR domain sequences as the antibody selected from hybridoma clones 4E12B2D10, 4E12B2, and 4E12.

### II. Examples of IL-12 Prodrugs

In some embodiments, the IL-12 prodrug comprises an antibody or an Fc domain, and an IL-12 agonist polypeptide; wherein the IL-12 agonist polypeptide is fused to the C-terminal of one of the heavy chains of the antibody or Fc polypeptide chain of the Fc domain, optionally through a peptide linker; wherein said IL-12 masking moiety is fused to the C-terminal of the 2^{nd} heavy chain or Fc polypeptide chain, through a cleavable peptide linker. Examples of the prodrugs are illustrated in FIGS. 1A, 1B, 2A, 2B, 3A, 3B, 4A, 4B, 5A, 5B, 6A, 6B, 7A, and 7B. All the prodrugs are shown with the cytokine components p35 and p40 and the masking moieties located on the C-termini of the Fc domains. In some embodiments, the cytokine components p35 and p40 and the masking moieties are located on the N-termini of the Fc domains. In addition, prodrugs may comprise both IL-12 and IL-2 agonist polypeptides or both IL-12 and IL-15 agonist polypeptides.

By way of example, an IL-12 prodrug comprises two polypeptide chains, wherein the first polypeptide chain comprises an amino acid sequence at least 99% identical to SEQ ID NO: 20; and the second polypeptide chain comprises an amino acid sequence at least 99% identical as on selected from SEQ ID NOs: 21-28.

By way of another example, the IL-12 prodrug comprises two polypeptide chains, wherein the first polypeptide chain comprises an amino acid sequence at least 99% identical to SEQ ID NO: 55; and said second polypeptide chain comprises an amino acid sequence at least 99% identical as on selected from SEQ ID NOs: 56-59.

By way of another example, the IL-12 prodrug comprises two polypeptide chains, wherein the first polypeptide chain comprises an amino acid sequence at least 99% identical to SEQ ID NO: 60; and the second polypeptide chain comprises an amino acid sequence at least 99% identical as on selected from SEQ ID NOs: 61-64.

By way of another example, the IL-12 prodrug comprises two polypeptide chains, wherein the first polypeptide chain comprises an amino acid sequence at least 99% identical to SEQ ID NO: 65; and the second polypeptide chain comprises an amino acid sequence at least 99% identical as on selected from SEQ ID NOs: 66-73.

By way of another example, the IL-12 prodrug comprises two polypeptide chains, wherein the first polypeptide chain comprises an amino acid sequence at least 99% identical to SEQ ID NO: 20; and the second polypeptide chain comprises an amino acid sequence at least 99% identical as on selected from SEQ ID NOs: 76-79.

In some embodiments, in the prodrugs that further comprise a second cytokine or effector moiety, the second cytokine moiety is an TL-2 agonist polypeptide, such as one with an amino acid sequence selected from SEQ ID NOs: 74 and 75. By way of example, the second cytokine may be fused to the N-terminus of one of the Fc domain polypeptides, while the IL-12 agonist polypeptides and masking moieties are fused to the C-termini of the Fc domains, as illustrated in FIGS. 1A-7B. In some embodiments, the IL-12 prodrugs may further comprise one or more masking moieties that bind to and inhibit a biological activity of the second cytokine.

### III. Pharmaceutical Compositions

Pharmaceutical compositions of the prodrugs are prepared by mixing the presently disclosed prodrugs, or antibody fusion molecules or the antibody fusion molecule drug conjugate having the desired degree of purity with one or more optional pharmaceutically acceptable carriers (see Osol, A. Ed. Remington's Pharmaceutical Sciences 16th edition (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may comprise as—or in addition to—the carrier, excipient or diluent any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes. In some embodiment, said formulation is administrated directly in a tumor or tumors.

In some embodiments, an antibody or protein formulation is a lyophilized formulation. In another embodiment, an antibody or protein formulation is an aqueous formulation.

In some embodiments, the pharmaceutical composition is a combination pharmaceutical composition, which comprises an IL-12 prodrug of the present disclosure, a pharmaceutically acceptable excipient, and a second active ingredient selected from a cytokine other than IL-12 or its fusion molecule, an antibody against PD-1, an antibody against PD-L1, an antibody against CTLA-4, an antibody against CD47, a PD-1 antibody-IL-15 fusion molecule, a PD-1-IL-2 fusion molecule, and a PD-1-IL-21 fusion molecule.

### IV. Methods of Treatment

The presently disclosed prodrugs can be used to treat a disease, depending on the antigen bound by the antigen-binding domain. In some embodiments, the prodrugs disclosed herein are used to treat cancer. In some embodiments, the prodrugs are used to treat an infection, for example when the drug molecule is an antibacterial agent or an antiviral agent.

In some embodiments, a method of treating a disease (such as cancer, a viral infection, or a bacterial infection) in a subject comprises administering to the subject an effective amount of the presently disclosed prodrugs.

In some embodiments, the cancer is a solid cancer. In some embodiments, the cancer is a blood cancer. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer, cholangiocarcinoma, breast cancer, and ovarian cancer.

In some embodiments, the presently disclosed prodrugs are used to treat a bacterial infection such as sepsis. In some embodiments, the bacteria causing the bacterial infection are drug-resistant bacteria. In some embodiments, the antigen-binding moiety binds to a bacterial antigen.

In some embodiments, the prodrug is used to treat a viral infection. In some embodiments, the virus causing the viral infection is hepatitis C (HCV), hepatitis B (HBV), human immunodeficiency virus (HIV), a human papilloma virus (HPV). In some embodiments, the antigen-binding moiety binds to a viral antigen.

Generally, dosages and routes of administration of the present pharmaceutical compositions are determined according to the size and condition of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the composition is administered to a subject intravenously.

In some embodiments, the prodrug is administered to a subject in need a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the antibody fusion molecule conjugated to the drug is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 2, 3, 4, 5, 7, 10, 15, or 20 or more doses).

In some embodiments, the IL-12 prodrug is administered to a subject in combination with a second pharmaceutical composition, wherein the second pharmaceutical composition comprises an active ingredient selected from a cytokine other than IL-12 or its fusion molecule, an antibody against PD-1, an antibody against PD-L1, an antibody against CTLA-4, an antibody against CD47, a PD-1 antibody-IL-2 fusion molecule, a PD-1-IL-7 fusion molecule, a PD-1 antibody-IL-15 fusion molecule, and a PD-1-IL-21 fusion molecule.

### V. Methods of Making the Prodrugs

The presently disclosed prodrugs can be produced using recombinant DNA methods. Nucleic acid molecules encoding the polypeptide or the fusion polypeptide of said prodrug can be isolated and inserted into one or more vectors for further cloning and/or expression in a host cell. Such nucleic acid molecules may be readily isolated and sequenced using conventional methods. Suitable host cells for cloning or expression of fusion polypeptide vectors include prokaryotic cells or eukaryotic cells. Exemplary host cells include Chinese Hamster Ovary (CHO) cells or human embryonic kidney cells (e.g., HEK293).

Expression host cells express the antibody fusion molecule. After an expression period, the host cells can by lysed and the prodrug or antibody fusion molecule can be purified. Exemplary purification methods include liquid chromatography, such as ion exchange chromatography, affinity chromatography (such as Protein A affinity chromatography), or size exclusion chromatography.

It is understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is not intended to be limited to a particular compound, composition, article, or method, unless expressly stated as such. In addition, those of ordinary skill in the art will recognize that certain changes, modification, permutations, alterations, additions, subtractions, and sub-combinations thereof can be made in accordance with the teachings herein without departing from the spirit of the present specification.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### VI. Exemplary Embodiments

Non-limiting, exemplary embodiments of the present disclosure are described below.

1. A Prodrug of IL-12 which comprises an IL-12 p40 subunit (p40), an IL-12 p35 subunit (p35), a first masking moiety (MM), a second masking moiety, a carrier (C), and at least one cleavable peptide linker; wherein:

a. Said IL-12 subunit p40 has an amino acid sequence at least 95% identical to that of SEQ ID NO: 5;

b. Said IL-12 subunit p35 has an amino acid sequence at least 95% identical to that of SEQ ID NO: 6;

c. Said first masking moiety comprises a binding moiety which binds to p40, and said second masking moiety comprises a binding moiety which binds to p35; and

d. Said carrier is selected from an albumin or albumin fragment, an Fc domain, and an antibody.

2. The prodrug of embodiment 1, wherein said first masking moiety is selected from the extracellular domain of IL-12 receptor R31 (IL-12Rβ1 ECD) or a fragment thereof, a p40-binding peptide discovered from screening of a peptide library, and a single chain Fv or Fab domain of an antibody which binds to p40.
3. The prodrug of embodiment 1 or 2, wherein said second masking moiety is selected from the extracellular domain of IL-12 receptor 32 (IL-12Rβ2 ECD) or a fragment thereof, a p35 or p40-binding peptide discovered from screening of a peptide library, and a single chain Fv or Fab domain of an antibody which binds to p35.
4. The prodrug of any of embodiments 1-3, wherein said first masking moiety comprises the extracellular domain (ECD) of IL-12 receptor beta1 (IL-12Rβ1) or a fragment thereof, wherein said IL-12Rβ1 ECD comprises an amino acid sequence at least 95% identical as that of SEQ ID NO: 13.
5. The prodrug of any of embodiments 1-3, wherein said first masking moiety comprises a fragment of IL-12Rβ1 ECD, wherein said fragment comprises an amino acid sequence at least 95% identical as that of SEQ ID NO: 14, 15, or 16.
6. The prodrug of any of embodiments 1-3, wherein said second masking moiety comprises the extracellular domain (ECD) of IL-12 receptor beta2 (IL-12Rβ2) or a fragment thereof, wherein said IL-12Rβ2 ECD comprises an amino acid sequence at least 95% identical as that of SEQ ID NO: 17.
7. The prodrug of any of embodiments 1-3, wherein said second masking moiety comprises a fragment of IL-12Rβ2 ECD, wherein said fragment comprises an amino acid sequence at least 95% identical as that of SEQ ID NOs: 18 or 19.
8. The prodrug according to any of embodiments 1-3, wherein said first masking moiety comprises a scFv which binds to p40, wherein said scFv comprises an amino acid sequence at least 99% identical as one selected from SEQ ID NOs: 7-11 and 12.
9. The prodrug of any of embodiments 1-3, wherein said first masking moiety is selected from the IL-12Rβ1 ECD or a fragment thereof comprising an amino acid sequence selected from SEQ ID NOs: 13-16, or a scFv with an amino acid sequence selected from SEQ ID NOs: 7-12; and wherein said second masking moiety comprises the IL-12Rβ2 ECD or a fragment thereof comprising an amino acid sequence selected from SEQ ID NOs: 17-19.
10. The prodrug of any of embodiments 1-3, wherein said first masking moiety is selected from the IL-12Rβ1 ECD or a fragment thereof comprising an amino acid sequence selected from SEQ ID NOs: 13-16, or a scFv with an amino acid sequence selected from SEQ ID NOs: 7-12; and wherein said second masking moiety comprises a scFv which binds to p35.
11. The prodrug of any of embodiments 1-10, wherein said carrier is an antibody which binds to a target expressed on the surface of an immune cell or a cancer cell.
12. The prodrug of any of embodiments 1-10, wherein it further comprises another cytokine or effector moiety.
13. The prodrug of embodiment 12, wherein said cytokine moiety comprises an IL-2 agonist polypeptide, which comprises an amino acid sequence selected from SEQ ID NOs: 74 and 75.
14. The prodrug of any of embodiments 1-10, wherein said carrier comprises an Fe domain which comprises a first polypeptide chain F1 and a second polypeptide chain F2, wherein said F1 and F2 forms heterodimerization; wherein said p40 or its analog is fused to the C-terminus of F1 optionally through a non-cleavable peptide linker, said p35 or its analog is fused to the C-terminus of p40 through a non-cleavable peptide linker; and wherein said first masking moiety is fused to the C-terminus of F2 through a cleavable or non-cleavable peptide linker, and said second masking moiety is fused to the C-terminus of the said first masking moiety through a cleavable peptide linker.
15. The prodrug of any of embodiments 1-10, wherein said carrier comprises an Fc domain which comprises a first polypeptide chain F1 and a second polypeptide chain F2, wherein said F1 and F2 forms heterodimerization; wherein said p40 is fused to the C-terminus of F1 optionally through a non-cleavable peptide linker, and said first masking moiety is fused to the C-terminus of p40 through a cleavable or non-cleavable peptide linker; and wherein said p35 is fused to the C-terminus of F2, and said second masking moiety is fused to the C-terminus of p35 through a cleavable peptide linker.
16. The prodrug of any of embodiments 1-10, wherein said carrier comprises an Fc domain which comprises a first polypeptide chain F1 and a second polypeptide chain F2, wherein said F1 and F2 forms heterodimerization; wherein said p35 is fused to the C-terminus of F1 optionally through a non-cleavable peptide linker, and said first masking moiety is fused to the C-terminus of p35 through a cleavable or non-cleavable peptide linker; and wherein said p40 is fused to the C-terminus of F2, and said second masking moiety is fused to the C-terminus of p40 through a cleavable peptide linker.
17. The prodrug of any of embodiments 1-16, wherein said non-cleavable peptide linker comprises an amino acid sequence from SEQ ID NOs: 29-33; and wherein said cleavable peptide linker comprises an amino acid sequence selected from SEQ ID NOs: 34-54.
18. The prodrug of embodiment 1 which comprises two polypeptide chains, wherein said first polypeptide chain comprises an amino acid sequence at least 99% identical as one selected from SEQ ID NO: 20; and said second polypeptide chain comprises an amino acid sequence at least 99% identical as on selected from SEQ ID NOs: 21-28.
19. The prodrug of embodiment 1 which comprises two polypeptide chains, wherein said first polypeptide chain comprises an amino acid sequence at least 99% identical as SEQ ID NO: 55; and said second polypeptide chain comprises an amino acid sequence at least 99% identical as on selected from SEQ ID NOs: 56-59.
20. The prodrug of embodiment 1 which comprises two polypeptide chains, wherein said first polypeptide chain comprises an amino acid sequence at least 99% identical as one selected from SEQ ID NO: 60; and said second polypeptide chain comprises an amino acid sequence at least 99% identical as on selected from SEQ ID NOs: 61-64.
21. The prodrug of embodiment 1 which comprises two polypeptide chains, wherein said first polypeptide chain comprises an amino acid sequence at least 99% identical as SEQ ID NO: 65; and said second polypeptide chain comprises an amino acid sequence at least 99% identical as on selected from SEQ ID NOs: 66-73.
22. A pharmaceutical composition comprising the prodrug of any one of embodiments 1-21 and a pharmaceutically acceptable excipient.
23. A polynucleotide or polynucleotides encoding the prodrug of any one of embodiments 1-22.
24. An expression vector or vectors comprising the polynucleotide or polynucleotides of embodiment 23.
25. A host cell comprising the vector(s) of embodiment 24.
26. The host cell of embodiment 25, wherein the host cell has the gene or genes encoding uPA, MMP-2, MMP-9 and/or matriptase are knocked out.
27. A method of making the prodrug of any one of embodiments 1-21, comprising culturing the host cell of claim 25 or 26 under conditions that allow expression of the antigen-binding molecule, and isolating the antigen-binding molecule.
28. A method of treating a cancer or an infectious disease or stimulating the immune system in a patient in need thereof, comprising administering the pharmaceutical composition of embodiment 22.
29. A prodrug for use in treating a cancer or an infectious disease or stimulating the immune system in the method of embodiment 28.
30. The method of embodiment 28, wherein said cancer is selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.
31. A method of treating of a patient with cancer, comprising administering the pharmaceutical composition of claim 22 directly into a tumor or tumors.

In order that this invention may be better understood, the following examples are set forth. These examples are for purposes of illustration only and are not to be construed as limiting the scope of the invention in any manner.

### EXAMPLES

### Example 1: Transient Transfection of the IL-12 Prodrugs Using HEK293 Cells

Expression plasmids were co-transfected into approximately 4×10⁶ cell/ml freestyle HEK293 cells at approximately 2.5 μg/ml using PEI (polyethylenimine). For Fc-based IL-12 prodrugs, the Fc-IL-12 fusion polypeptide and the Fc-masking moiety fusion polypeptide were in a 1:2 ratio. The cell cultures were harvested 6 days after transfection by centrifuging at 9,000 rpm for 45 min followed by 0.22 μM filtration. A number of IL-12 prodrugs were expressed. The Sequence ID NOs are listed in Table 2.

TABLE 2
Sequence Information of the samples.

| | SEQ ID for | SEQ ID for |
|---|---|---|
| | Fc-IL-12 | Fc-Masking |
| Prodrug or | Fusion | Moiety Fusion |
| Control Name | Polypeptide | Polypeptide |
| JR3.105.2 | SEQ ID NO: 20 | SEQ ID NO: 76 |
| JR3.105.3 | SEQ ID NO: 20 | SEQ ID NO: 77 |
| JR3.105.4 | SEQ ID NO: 20 | SEQ ID NO: 78 |
| JR3.105.5 | SEQ ID NO: 20 | SEQ ID NO: 79 |

### Example 2: Purification of the Fc-Based IL-12 Prodrugs

The purifications of the proteins of the Fc-based IL-12 prodrugs were carried out by using Protein A Affinity. Briefly, the supernatant of the transient expression cell culture was loaded onto a Protein A column, which was equilibrated with 25 mM Tris-HCl, 30 mM NaCl, pH 7.8 (buffer A) before applying the sample. The column was washed with 5-column volumes of buffer A and the bound protein was eluted with 50 mM acetic acid, pH 3.6. The pH of the eluted protein was adjusted to approximately 5.2 using 1 M Tris base. The samples were analyzed by SDS-PAGE as described below.

### Example 3: SDS-PAGE Analysis

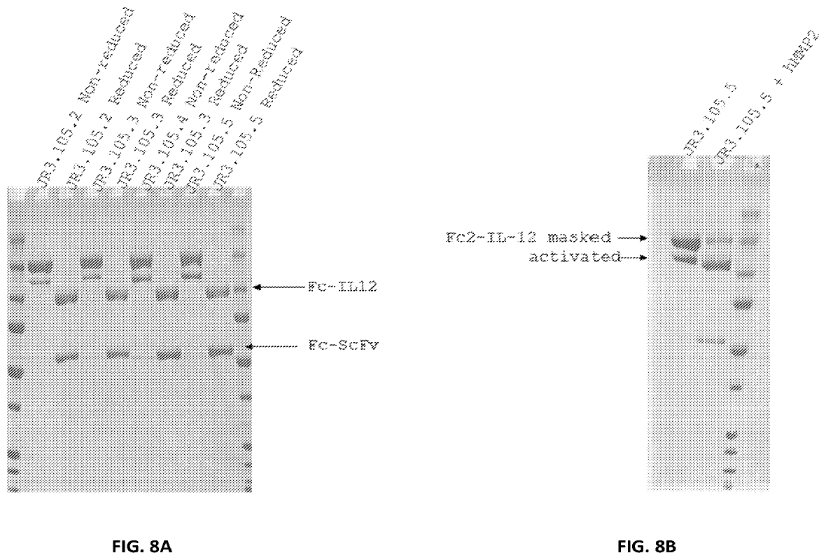

10 μl of the 10-20 μg of purified protein samples were mixed with Bolt™ LDS Sample Buffer (Novex) with or without reducing reagents. The samples were heated at 70° C. for 3 min and then loaded to a NuPAGE™ 4-12% BisTris Gel (Invitrogen™). The gel was run in NuPAGE™ MOPS SDS Running buffer (Invitrogen™) at 200 Volts for 40 min and then stained with Coomassie. The purified samples of prodrugs were analyzed by the SDS-PAGE analysis, as shown in FIG. 8A. The JR3.105.5 (with a structure as illustrated in FIG. 5B) sample treated with the protease MMP-2 (see below) was also analyzed by the SDS-PAGE analysis, as shown in FIG. 8B. The data show that the masking moieties of the prodrugs were largely removed by the protease digestion, and that the activated molecules migrated at the expected molecular weights. In addition, it appeared that the Protein A column pool sample also contained activated version of the IL-12 prodrug (FIG. 8B).

### Example 4: Proteolytic Treatment

The proteases, human MM1P2, human MMP9, mouse MMP2 and mouse MMP9 were purchased from R&D systems. The protease digestion was carried out by incubating 10 μg-50 μg of prodrugs with 1 μg of human MMP2, human MMP9, mouse MMP2 or mouse MMP9 in the HBS buffer (20 mM HEPES, 150 mM NaCl₂, pH 7.4) containing 2 mM CaCl₂) and 10 μM ZnCl₂ at 37° C. for 12 hours. The prodrug JR3.105.5 prior to and after digestion were analyzed by SDS-PAGE (FIG. 8B) and the cell-based activity assay (see below).

### Example 5: Cell-Based IL-12 Reporter Assay

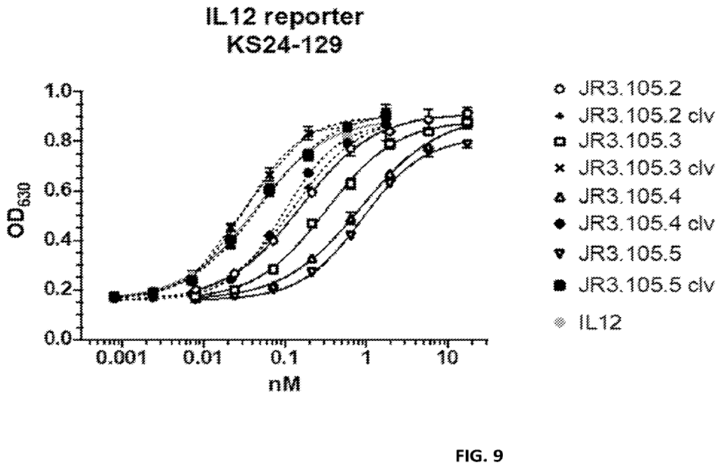

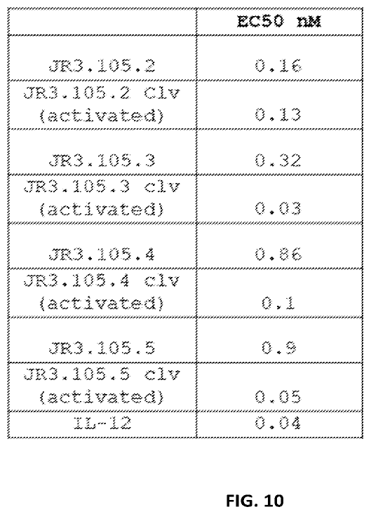

IL-12 prodrugs prior to and after activation was assayed for biological activity using a secreted alkaline phosphatase reporter cell line (HEK-Blue IL-12, InvivoGen catalog # hkb-i112). The HEK-Blue IL-12 cell line was generated through the stable introduction of the genes for the human IL-12 receptor and signaling pathway into HEK 293 cells. The cells also express a STAT4 inducible secreted alkaline phosphatase (SEAP) reporter gene. Test articles are diluted in 100 μL/well culture medium in 96 well flat bottom cell culture plates, followed by addition of HEK-Blue IL-12 cells at 50,000 cells/well in 100 μL. Cultures are incubated at 37° C. overnight. 20 μL of culture supernatant is transferred to an ELISA plate, and 180 μL Quanti-Blue (InvivoGen, Catalog #rep-qb1) is added. The ELISA plate is incubated at 37° C. for 1 hour, and OD₆₃₀ is measured with a microplate reader. The data are shown in FIG. 9 and FIG. 10. The results show that JR3.105.5 had approximately an 18-fold activation based on the EC₅₀ values (FIG. 10). As shown in FIG. 8B, IL-12 prodrug JR3.105.5 sample may have contained a small amount of the activated version of the prodrug. Consequently, the masking efficiency of the masking moiety of JR3.105.5 may have been stronger than was shown in FIGS. 9 and 10.

This IL-12 prodrug JR3.105.5 also had the highest activation compared with the other samples. The IL-12 prodrugs all had the same Fc-IL-12 chain (SEQ ID NO: 20, Table 2). However, the masking moiety of JR3.105.5 had the longest linker between its VL and VH, comprising (G4S)₄, as shown SEQ ID NO: 80.

### Example 6: In Vivo Efficacy Study with a Mouse Tumor Model

The in vivo anti-tumor effect of the IL-12-Fc prodrugs of the invention is tested in a mouse model. NOD SCID gamma (NSG) mice are engrafted intradermally with 3×10⁶ pp-65 expressing MCF-7 cells in the right flank. After the tumors are established, mice are engrafted intraperitoneally with 1.5×10⁶ human PBMCs. Mice are then treated on twice weekly, weekly, or biweekly with the IL-12 prodrugs. Tumor volumes and body weights are measured every other day. The mice are sacrificed at the end of the study, and blood samples, serum samples, and tissues (tumors and major organs including livers and lungs) are analyzed for PK, safety, cytokine release, and prodrug activation.

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

### LIST OF SEQUENCES

human IL-2: YKNPKLTRML TFKFYMPKKA TELKHLQGLE EELKPLEEVL
SEQ ID NO: 1
APTSSSTKKT QLQLEHLLLD LQMILNGINN TTFMCEYADE TATIVEFLNR WITFCQSIIS TLT
NLAQSKNFHL RPRDLISNIN VIVLELKGSE
Human IL-2 Receptor Beta Subunit EXtracellular Domain
(https://www.uniprot.4 org/uniprot/P14784)
SEQ ID NO: 2
AVNGTSQFTC FYNSRANISC VWSQDGALQDTSCQVHAWPDRRRVVNQTCELLPVSQASWACNLILGAPDSQ
KLTTVDIVTL RVLCREGVRW RVMAIQDFKPFENLRLMAPISLQWHVETHRCNISWEISQASHYFERHLE
FEARTLSPGH TWEEAPLLTL KQKQEWICLETLTPDTQYEFQVRVKPLQGEFTTWSPWSQPLAFRTKPAAL
GKDT
IL15 receptor alpha subunit sushi domain LKCIRDPALV
SEQ ID NO: 3
ITCPPPMSVE HADIWVKSYS LYSRERYICN SGFKRKAGTS SLTECVLNKA TNVAHWTTPS
HQRPAPP
human IL15 IEDLIQSMHI DATLYTESDV HPSCKVTAMK CFLLELQVIS
SEQ ID NO: 4
GIHVFILGCF SAGLPKTEAN WVNVISDLKK ESGCKECEEL EEKNIKEFLQ SFVHIVQMFI NTS
LESGDASIHD TVENLIILAN NSLSSNGNVT
human IL-12 p40 subunit DQSSEVLGSG KTLTIQVKEF GDAGQYTCHK
SEQ ID NO: 5
IWELKKDVYV VELDWYPDAP GEMVVLTCDT PEEDGITWTL AKNYSGRFTC WWLTTISTDL TFSVKSSRGS
GGEVLSHSLL LLHKKEDGIW STDILKDQKE PKNKTFLRCE EESLPIEVMV DAVHKLKYEN YTSSFFIRDI
SDPQGVTCGA ATLSAERVRG DNKEYEYSVE CQEDSACPAA FCVQVQGKSK REKKDRVFTD KTSATVICRK
IKPDPPKNLQ LKPLKNSRQV EVSWEYPDTW STPHSYFSLT
NASISVRAQD RYYSSSWSEW ASVPCS
human IL-12 p35 subunit PCTSEEIDHE DITKDKTSTV EACLPLELTK
SEQ ID NO: 6
RNLPVATPDP GMFPCLHHSQ NLLRAVSNML QKARQTLEFY MYQVEFKTMN AKLLMDPKRQ IFLDQNMLAV
NESCLNSRET SFITNGSCLA SRKTSFMMAL CLSSIYEDLK RIRAVTIDRV MSYLNAS
IDELMQALNF NSETVPQKSS LEEPDFYKTK IKLCILLHAF
scFv which binds to IL-12 PGKGLDWIGI MSPVDSDIRY SPSFQGQVTM
SEQ ID NO: 7
EVQLVQSGAE VKKPGESLKI SCKGSGYSFT TYWLGWVRQM QGTLVTVSSG GGGSGGGGSD IQMTQSPSSL
SVDKSITTAY LQWNSLKASD TAMYYCARRR PGQGYFDFWG SSLQSGVPSR FSGSGSGTDF TLTISSLQPE
SASVGDRVTI TCRASQGISS WLAWYQQKPE KAPKSLIYAA
DFATYYCQQY NIYPYTFGQG TKLEIK
scFv which binds to IL-12
SEQ ID NO: 8
DIQMTQSPSS LSASVGDRVT ITCRASQGIS SWLAWYQQKP EKAPKSLIYA ASSLQSGVPS RFSGSGSGTD
FTLTISSLQP EDFATYYCQQ YNIYPYTFGQ GTKLEIKGGG GSGGGGSEVQ LVQSGAEVKK PGESLKISCK
GSGYSFTTYW LGWVRQMPGK GLDWIGIMSP VDSDIRYSPS FQGQVTMSVD KSITTAYLQW NSLKASDTAM
YYCARRRPGQ GYFDFWGQGT LVTVSS
scFv which binds to IL-12
SEQ ID NO: 9
EVQLVQSGAE VKKPGESLKI SCKGSGYSFT TYWLGWVRQM PGKGLDWIGI MSPVDSDIRY SPSFQGQVTM
SVDKSITTAY LQWNSLKASD TAMYYCARRR PGQGYFDFWG QGTLVTVSSG GGGSGGGGSG GGGSDIQMTQ
SPSSLSASVG DRVTITCRAS QGISSWLAWY QQKPEKAPKS LIYAASSLQS GVPSRFSGSG SGTDFTLTIS
SLQPEDFATY YCQQYNIYPY TFGQGTKLEI K
scFv which binds to IL-12
SEQ ID NO: 10
DIQMTQSPSS LSASVGDRVT ITCRASQGIS SWLAWYQQKP EKAPKSLIYA ASSLQSGVPS RFSGSGSGTD
FTLTISSLQP EDFATYYCQQ YNIYPYTFGQ GTKLEIKGGG GSGGGGSGGG GSEVQLVQSG AEVKKPGESL
KISCKGSGYS FTTYWLGWVR QMPGKGLDWI GIMSPVDSDI RYSPSFQGQV TMSVDKSITT AYLQWNSLKA
SDTAMYYCAR RRPGQGYFDF WGQGTLVTVS S
scFv which binds to IL-12
SEQ ID NO: 11
EVQLVQSGAE VKKPGESLKI SCKGSGYSFT TYWLGWVRQM PGKGLDWIGI MSPVDSDIRY SPSFQGQVTM
SVDKSITTAY LQWNSLKASD TAMYYCARRR PGQGYFDFWG QGTLVTVSSG GGGSGGGGSG GGGSGGGGSD
IQMTQSPSSL SASVGDRVTI TCRASQGISS WLAWYQQKPE KAPKSLIYAA SSLQSGVPSR FSGSGSGTDF
TLTISSLQPE DFATYYCQQY NIYPYTFGQG TKLEIK
scFv which binds to IL-12
SEQ ID NO: 12
DIQMTQSPSS LSASVGDRVT ITCRASQGIS SWLAWYQQKP EKAPKSLIYA ASSLQSGVPS RFSGSGSGTD
FTLTISSLQP EDFATYYCQQ YNIYPYTFGQ GTKLEIKGGG GSGGGGSGGG GSGGGGSEVQ LVQSGAEVKK
PGESLKISCK GSGYSFTTYW LGWVRQMPGK GLDWIGIMSP VDSDIRYSPS FQGQVTMSVD KSITTAYLQW
NSLKASDTAM YYCARRRPGQ GYFDFWGQGT LVTVSS
IL-12 receptor beta 1 eXtracellular domain (IL-12R1 ECD)
SEQ ID NO: 13
RTSECCFQDP PYPDADSGSA SGPRDLRCYR ISSDRYECSW QYEGPTAGVS HFLRCCLSSG
RCCYFAAGSA TRLQFSDQAG VSVLYTVTLW VESWARNQTE KSPEVTLQLY NSVKYEPPLG
DIKVSKLAGQ LRMEWETPDN QVGAEVQFRH RTPSSPWKLG DCGPQDDDTE SCLCPLEMNV
AQEFQLRRRQ LGSQGSSWSK WSSPVCVPPE NPPQPQVRFS VEQLGQDGRR RLTLKEQPTQ
LELPEGCQGL APGTEVTYRL QLHMLSCPCK AKATRTLHLG KMPYLSGAAY NVAVISSNQF
GPGLNQTWHI PADTHTEPVA LNISVGTNGT TMYWPARAQS MTYCIEWQPV GQDGGLATCS
LTAPQDPDPA GMATYSWSRE SGAMGQEKCY YITIFASAHP EKLTLWSTVL STYHFGGNAS
AAGTPHHVSV KNHSLDSVSV DWAPSLLSTC PGVLKEYWR CRDEDSKQVS EHPVQPTETQ
VTLSGLRAGV AYTVQVRADT AWLRGVWSQP QRFSIEVQVS D
IL-12Rβ1 ECD D1-D5
SEQ ID NO: 14
RTSECCFQDP PYPDADSGSA SGPRDLRCYR ISSDRYECSW QYEGPTAGVS HFLRCCLSSG
RCCYFAAGSA TRLQFSDQAG VSVLYTVTLW VESWARNQTE KSPEVTLQLY NSVKYEPPLG
DIKVSKLAGQ LRMEWETPDN QVGAEVQFRH RTPSSPWKLG DCGPQDDDTE SCLCPLEMNV
AQEFQLRRRQ LGSQGSSWSK WSSPVCVPPE NPPQPQVRFS VEQLGQDGRR RLTLKEQPTQ
LELPEGCQGL APGTEVTYRL QLHMLSCPCK AKATRTLHLG KMPYLSGAAY NVAVISSNQF
GPGLNQTWHI PADTHTEPVA LNISVGTNGT TMYWPARAQS MTYCIEWQPV GQDGGLATCS
LTAPQDPDPA GMATYSWSRE SGAMGQEKCY YITIFASAHP EKLTLWSTVL STYHFGGNAS
AAGTPHHVSV KNHSLDSVSV DWAPSLLSTC PGVLKEYWR CRDEDSKQVS EHPVQPTETQ
VTLSGLRAGV AYTVQVRADT AWLRGVWSQP QRFSIEVQVS D
IL-12Rβ1 ECD D1-D2
SEQ ID NO: 15
RTSECCFQDP PYPDADSGSA SGPRDLRCYR ISSDRYECSW QYEGPTAGVS HFLRCCLSSG
RCCYFAAGSA TRLQFSDQAG VSVLYTVTLW VESWARNQTE KSPEVTLQLY NSVKYEPPLG
DIKVSKLAGQ LRMEWETPDN QVGAEVQFRH RTPSSPWKLG DCGPQDDDTE SCLCPLEMNV
AQEFQLRRRQ LGSQGSSWSK WSSPVCVPPE
IL-12Rβ1 ECD D1-D3
SEQ ID NO: 16
RTSECCFQDP PYPDADSGSA SGPRDLRCYR ISSDRYECSW QYEGPTAGVS HFLRCCLSSG
RCCYFAAGSA TRLQFSDQAG VSVLYTVTLW VESWARNQTE KSPEVTLQLY NSVKYEPPLG
DIKVSKLAGQ LRMEWETPDN QVGAEVQFRH RTPSSPWKLG DCGPQDDDTE SCLCPLEMNV
AQEFQLRRRQ LGSQGSSWSK WSSPVCVPPE NPPQPQVRFS VEQLGQDGRR RLTLKEQPTQ
LELPEGCQGL APGTEVTYRL QLHMLSCPCK AKATRTLHLG KMPYLSGAAY NVAVISSNQF
GPGLNQTWHI PA
IL-12Rβ2 ECD D1-D6
SEQ ID NO: 17
KIDACKRGDV TVKPSHVILL GSTVNITCSL KPRQGCFHYS RRNKLILYKF DRRINFHHGH
SLNSQVTGLP LGTTLFVCKL ACINSDEIQI CGAEIFVGVA PEQPQNLSCI QKGEQGTVAC
TWERGRDTHL YTEYTLQLSG PKNLTWQKQC KDIYCDYLDF GINLTPESPE SNFTAKVTAV
NSLGSSSSLP STFTFLDIVR PLPPWDIRIK FQKASVSRCT LYWRDEGLVL LNRLRYRPSN
SRLWNMVNVT KAKGRHDLLD LKPFTEYEFQ ISSKLHLYKG SWSDWSESLR AQTPEEEPTG
MLDVWYMKRH IDYSRQQISL FWKNLSVSEA RGKILHYQVT LQELTGGKAM TQNITGHTSW
TTVIPRTGNW AVAVSAANSK GSSLPTRINI MNLCEAGLLA PRQVSANSEG MDNILVTWQP
PRKDPSAVQE YVVEWRELHP GGDTQVPLNW LRSRPYNVSA LISENIKSYI CYEIRVYALS
GDQGGCSSIL GNSKHKAPLS GPHINAITEE KGSILISWNS IPVQEQMGCL LHYRIYWKER
DSNSQPQLCE IPYRVSQNSH PINSLQPRVT YVLWMTALTA AGESSHGNER EFCLQGKAN
IL-12Rβ2 ECD D1-D2
SEQ ID NO: 18
KIDACKRGDV TVKPSHVILL GSTVNITCSL KPRQGCFHYS RRNKLILYKF DRRINFHHGH
SLNSQVTGLP LGTTLFVCKL ACINSDEIQI CGAEIFVGVA PEQPQNLSCI QKGEQGTVAC
TWERGRDTHL YTEYTLQLSG PKNLTWQKQC KDIYCDYLDF GINLTPESPE SNFTAKVTAV
NSLGSSSSLP STFTFLDI
IL-12Rβ2 ECD D1-D3
SEQ ID NO: 19
KIDACKRGDV TVKPSHVILL GSTVNITCSL KPRQGCFHYS RRNKLILYKF DRRINFHHGH
SLNSQVTGLP LGTTLFVCKL ACINSDEIQI CGAEIFVGVA PEQPQNLSCI QKGEQGTVAC
TWERGRDTHL YTEYTLQLSG PKNLTWQKQC KDIYCDYLDF GINLTPESPE SNFTAKVTAV
NSLGSSSSLP STFTFLDIVR PLPPWDIRIK FQKASVSRCT LYWRDEGLVL LNRLRYRPSN
SRLWNMVNVT KAKGRHDLLD LKPFTEYEFQ ISSKLHLYKG SWSDWSESLR AQTPEEEPTG
Fc IL-12 (P40-P35) with knobs mutations
SEQ ID NO: 20
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSIWELKKDV YVVELDWYPD APGEMVVLTC DTPEEDGITW
TLDQSSEVLG SGKTLTIQVK EFGDAGQYTC HKGGEVLSHS LLLLHKKEDG IWSTDILKDQ KEPKNKTFLR
CEAKNYSGRF TCWWLTTIST DLTFSVKSSR GSSDPQGVTC GAATLSAERV RGDNKEYEYS VECQEDSACP
AAEESLPIEV MVDAVHKLKY ENYTSSFFIR DIIKPDPPKN LQLKPLKNSR QVEVSWEYPD TWSTPHSYFS
LTFCVQVQGK SKREKKDRVF TDKTSATVIC RKNASISVRA QDRYYSSSWS EWASVPCSGG GGSGGGGSGG
GGSRNLPVAT PDPGMFPCLH HSQNLLRAVS NMLQKARQTL EFYPCTSEEI DHEDITKDKT STVEACLPLE
LTKNESCLNS RETSFITNGS CLASRKTSFM MALCLSSIYE DLKMYQVEFK TMNAKLLMDP KRQIFLDQNM
LAVIDELMQA LNFNSETVPQ KSSLEEPDFY KTKIKLCILL HAFRIRAVTI DRVMSYLNAS
Fc with hole mutations- IL-12Rβ1-D1D2/IL-12Rβ2-D1D2
SEQ ID NO: 21
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS RTSECCFQDP PYPDADSGSA SGPRDLRCYR ISSDRYECSW
QYEGPTAGVS HFLRCCLSSG RCCYFAAGSA TRLQFSDQAG VSVLYTVTLW VESWARNQTE KSPEVTLQLY
NSVKYEPPLG DIKVSKLAGQ LRMEWETPDN QVGAEVQFRH RTPSSPWKLG DCGPQDDDTE SCLCPLEMNV
AQEFQLRRRQ LGSQGSSWSK WSSPVCVPPE (GGGGS)n₁V HMPLGFLGPR QARVVN(GGG GS)n₂KIDAC
KRGDVTVKPS HVILLGSTVN ITCSLKPRQG CFHYSRRNKL ILYKFDRRIN FHHGHSLNSQ VTGLPLGTTL
FVCKLACINS DEIQICGAEI FVGVAPEQPQ NLSCIQKGEQ GTVACTWERG RDTHLYTEYT LQLSGPKNLT
WQKQCKDIYC DYLDFGINLT PESPESNFTA KVTAVNSLGS SSSLPSTFTF LDI; wherein n1 = 1,
2, 3, or 4, and n2 = 1, 2, 3, or 4.
Fc with hole mutations IL-12R1-D1D2D3/IL-12R2-D1D2D3
SEQ ID NO: 22
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS RTSECCFQDP PYPDADSGSA SGPRDLRCYR ISSDRYECSW
QYEGPTAGVS HFLRCCLSSG RCCYFAAGSA TRLQFSDQAG VSVLYTVTLW VESWARNQTE KSPEVTLQLY
NSVKYEPPLG DIKVSKLAGQ LRMEWETPDN QVGAEVQFRH RTPSSPWKLG DCGPQDDDTE SCLCPLEMNV
AQEFQLRRRQ LGSQGSSWSK WSSPVCVPPE NPPQPQVRFS VEQLGQDGRR RLTLKEQPTQ LELPEGCQGL
APGTEVTYRL QLHMLSCPCK AKATRTLHLG KMPYLSGAAY NVAVISSNQF GPGLNQTWHI PA(GGGGS)n₁
VHMPLGFLGP RQARVVN(GG GGS)n₂KIDA CKRGDVTVKP SHVILLGSTV NITCSLKPRQ GCFHYSRRNK
LILYKFDRRI NFHHGHSLNS QVTGLPLGTT LFVCKLACIN SDEIQICGAE IFVGVAPEQP QNLSCIQKGE
QGTVACTWER GRDTHLYTEY TLQLSGPKNL TWQKQCKDIY CDYLDFGINL TPESPESNFT AKVTAVNSLG
SSSSLPSTFT FLDIVRPLPP WDIRIKFQKA SVSRCTLYWR DEGLVLLNRL RYRPSNSRLW NMVNVTKAKG
RHDLLDLKPF TEYEFQISSK LHLYKGSWSD WSESLRAQTP EEEPTG;
n1 = 1, 2, 3, or 4, and n2 = 1, 2, 3, or 4.
Fc with hole mutations- IL-12R1-D1D2/IL-12R2-D1D2
SEQ ID NO: 23
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSRTSECCF QDPPYPDADS GSASGPRDLR
CYRISSDRYE CSWQYEGPTA GVSHFLRCCL SSGRCCYFAA GSATRLQFSD QAGVSVLYTV TLWVESWARN
QTEKSPEVTL QLYNSVKYEP PLGDIKVSKL AGQLRMEWET PDNQVGAEVQ FRHRTPSSPW KLGDCGPQDD
DTESCLCPLE MNVAQEFQLR RRQLGSQGSS WSKWSSPVCV PPE(GGGGS) ₙ₁VHMPLGFL GPRQARVVN(
GGGGS)n₂KI DACKRGDVTV KPSHVILLGS TVNITCSLKP RQGCFHYSRR NKLILYKFDR RINFHHGHSL
NSQVTGLPLG TTLFVCKLAC INSDEIQICG AEIFVGVAPE QPQNLSCIQK GEQGTVACTW ERGRDTHLYT
EYTLQLSGPK NLTWQKQCKD IYCDYLDFGI NLTPESPESN FTAKVTAVNS LGSSSSLPST FTFLDI;
wherein n1 = 1, 2, 3, or 4, and n2 = 1, 2, 3, or 4.
Fc with hole mutations IL-12R1-D1D2D3/IL-12R2-D1D2D3
SEQ ID NO: 24
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSRTSECCF QDPPYPDADS GSASGPRDLR
CYRISSDRYE CSWQYEGPTA GVSHFLRCCL SSGRCCYFAA GSATRLQFSD QAGVSVLYTV TLWVESWARN
QTEKSPEVTL QLYNSVKYEP PLGDIKVSKL AGQLRMEWET PDNQVGAEVQ FRHRTPSSPW KLGDCGPQDD
DTESCLCPLE MNVAQEFQLR RRQLGSQGSS WSKWSSPVCV PPENPPQPQV RFSVEQLGQD GRRRLTLKEQ
PTQLELPEGC QGLAPGTEVT YRLQLHMLSC PCKAKATRTL HLGKMPYLSG AAYNVAVISS NQFGPGLNQT
WHIPA(GGGG S)n₁VHMPLG FLGPRQARVV N(GGGGS)n₂ KIDACKRGDV TVKPSHVILL GSTVNITCSL
KPRQGCFHYS RRNKLILYKF DRRINFHHGH SLNSQVTGLP LGTTLFVCKL ACINSDEIQI CGAEIFVGVA
PEQPQNLSCI QKGEQGTVAC TWERGRDTHL YTEYTLQLSG PKNLTWQKQC KDIYCDYLDF GINLTPESPE
SNFTAKVTAV NSLGSSSSLP STFTFLDIVR PLPPWDIRIK FQKASVSRCT LYWRDEGLVL LNRLRYRPSN
SRLWNMVNVT KAKGRHDLLD LKPFTEYEFQ ISSKLHLYKG SWSDWSESLR AQTPEEEPTG;
n1 = 1, 2, 3, or 4, and n2 = 1, 2, 3, or 4.
Fc with hole mutations scFv/IL-12R2-D1D2D3
SEQ ID NO: 25
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS DIQMTQSPSS LSASVGDRVT ITCRASQGIS SWLAWYQQKP
EKAPKSLIYA ASSLQSGVPS RFSGSGSGTD FTLTISSLQP EDFATYYCQQ YNIYPYTFGQ GTKLEIKGGG
GSGGGGSGGG GSEVQLVQSG AEVKKPGESL KISCKGSGYS FTTYWLGWVR QMPGKGLDWI GIMSPVDSDI
RYSPSFQGQV TMSVDKSITT AYLQWNSLKA SDTAMYYCAR RRPGQGYFDF WGQGTLVTVS S(GGGGS)n₁
VHMPLGFLGP RQARVVN(GG GGS)n₂KIDA CKRGDVTVKP SHVILLGSTV NITCSLKPRQ GCFHYSRRNK
LILYKFDRRI NFHHGHSLNS QVTGLPLGTT LFVCKLACIN SDEIQICGAE IFVGVAPEQP QNLSCIQKGE
QGTVACTWER GRDTHLYTEY TLQLSGPKNL TWQKQCKDIY CDYLDFGINL TPESPESNFT AKVTAVNSLG
SSSSLPSTFT FLDIVRPLPP WDIRIKFQKA SVSRCTLYWR DEGLVLLNRL RYRPSNSRLW NMVNVTKAKG
RHDLLDLKPF TEYEFQISSK LHLYKGSWSD WSESLRAQTP EEEPTG;
n1 = 1, 2, 3, or 4, and n2 = 1, 2, 3, or 4.
Fc with hole mutations scFv/IL-12R2-D1D2D3
SEQ ID NO: 26
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSDIQMTQS PSSLSASVGD RVTITCRASQ
GISSWLAWYQ QKPEKAPKSL IYAASSLQSG VPSRFSGSGS GTDFTLTISS LQPEDFATYY CQQYNIYPYT
FGQGTKLEIK GGGGSGGGGS GGGGSEVQLV QSGAEVKKPG ESLKISCKGS GYSFTTYWLG WVRQMPGKGL
DWIGIMSPVD SDIRYSPSFQ GQVTMSVDKS ITTAYLQWNS LKASDTAMYY CARRRPGQGY FDFWGQGTLV
TVSS(GGGGS )n₁VHMPLGF LGPRQARVVN (GGGGS)n₂K IDACKRGDVT VKPSHVILLG STVNITCSLK
PRQGCFHYSR RNKLILYKFD RRINFHHGHS LNSQVTGLPL GTTLFVCKLA CINSDEIQIC GAEIFVGVAP
EQPQNLSCIQ KGEQGTVACT WERGRDTHLY TEYTLQLSGP KNLTWQKQCK DIYCDYLDFG INLTPESPES
NFTAKVTAVN SLGSSSSLPS TFTFLDIVRP LPPWDIRIKF QKASVSRCTL YWRDEGLVLL NRLRYRPSNS
RLWNMVNVTK AKGRHDLLDL KPFTEYEFQI SSKLHLYKGS WSDWSESLRA QTPEEEPTG;
n1 = 1, 2, 3, or 4, and n2 = 1, 2, 3, or 4.
Ec with hole mutations scFv/IL-12R2-D1D2D3
SEQ ID NO: 27
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS EVQLVQSGAE VKKPGESLKI SCKGSGYSFT TYWLGWVRQM
PGKGLDWIGI MSPVDSDIRY SPSFQGQVTM SVDKSITTAY LQWNSLKASD TAMYYCARRR PGQGYFDFWG
QGTLVTVSSG GGGSGGGGSG GGGSDIQMTQ SPSSLSASVG DRVTITCRAS QGISSWLAWY QQKPEKAPKS
LIYAASSLQS GVPSRFSGSG SGTDFTLTIS SLQPEDFATY YCQQYNIYPY TFGQGTKLEI K(GGGGS)n₁
VHMPLGFLGP RQARVVN(GG GGS)n₂KIDA CKRGDVTVKP SHVILLGSTV NITCSLKPRQ GCFHYSRRNK
LILYKFDRRI NFHHGHSLNS QVTGLPLGTT LFVCKLACIN SDEIQICGAE IFVGVAPEQP QNLSCIQKGE
QGTVACTWER GRDTHLYTEY TLQLSGPKNL TWQKQCKDIY CDYLDFGINL TPESPESNFT AKVTAVNSLG
SSSSLPSTFT FLDIVRPLPP WDIRIKFQKA SVSRCTLYWR DEGLVLLNRL RYRPSNSRLW NMVNVTKAKG
RHDLLDLKPF TEYEFQISSK LHLYKGSWSD WSESLRAQTP EEEPTG; n1 = 1, 2, 3, or 4, and n2
= 1, 2, 3, or 4.
Fc with hole mutations scFv/IL-12R2-D1D2D3
SEQ ID NO: 28
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSEVQLVQS GAEVKKPGES LKISCKGSGY
SFTTYWLGWV RQMPGKGLDW IGIMSPVDSD IRYSPSFQGQ VTMSVDKSIT TAYLQWNSLK ASDTAMYYCA
RRRPGQGYFD FWGQGTLVTV SSGGGGSGGG GSGGGGSDIQ MTQSPSSLSA SVGDRVTITC RASQGISSWL
AWYQQKPEKA PKSLIYAASS LQSGVPSRFS GSGSGTDFTL TISSLQPEDF ATYYCQQYNI YPYTFGQGTK
LEIK(GGGGS ) ₙ₁VHMPLGF LGPRQARVVN (GGGGS)n₂K IDACKRGDVT VKPSHVILLG STVNITCSLK
PRQGCFHYSR RNKLILYKFD RRINFHHGHS LNSQVTGLPL GTTLFVCKLA CINSDEIQIC GAEIFVGVAP
EQPQNLSCIQ KGEQGTVACT WERGRDTHLY TEYTLQLSGP KNLTWQKQCK DIYCDYLDFG INLTPESPES
NFTAKVTAVN SLGSSSSLPS TFTFLDIVRP LPPWDIRIKF QKASVSRCTL YWRDEGLVLL NRLRYRPSNS
RLWNMVNVTK AKGRHDLLDL KPFTEYEFQI SSKLHLYKGS WSDWSESLRA QTPEEEPTG; n1 = 1,
2, 3, or 4, and n2 = 1, 2, 3, or 4.
SEQ ID NO: 29-33 Peptide Linker
(SEQ ID NO: 29)
GGGGS
(SEQ ID NO: 30)
GGGGSGGGGS
(SEQ ID NO: 31)
GGGGSGGGGS GGGGS
(SEQ ID NO: 32)
GGGGSGGGGX GGGGSGGGGS, X = A or N
(SEQ ID NO: 33)
(GGGGSGGGGX₁ GGGGX₂GGGGS)ₙ, X₁ = S, A or N, X₂ = A or N, and
n = 1, 2, or 3.
cleavable peptide linker
SEQ ID NO: 34
GGSLSGRSDN HGGGGS
cleavable peptide linker
SEQ ID NO: 35
GGSLSGRSDN HGS
cleavable linker
SEQ ID NO: 36
GGGGSGGGGS GGGGSISSGL LSSGGSGGSL SGRSDNHGGG GS
MMP-2/MMP-9 Cleavable peptide linkers
SEQ ID NO: 37-43
(SEQ ID NO: 37)
GPLGVR
(SEQ ID NO: 38)
PLGMWSR
(SEQ ID NO: 39)
PLGLWAR
(SEQ ID NO: 40)
PQGIAGQR
(SEQ ID NO: 41)
PLGLAG
(SEQ ID NO: 42)
LALGPR
(SEQ ID NO: 43)
GGPLGMLSQS
urokinase plasminogen activator (uPA) Cleavable peptide linkers
SEQ ID NO: 44-52
(SEQ ID NO: 44)
GGGGRRGGS
(SEQ ID NO: 45)
TGRGPSWV
(SEQ ID NO: 46)
SARGPSRW
(SEQ ID NO: 47)
TARGPSEK
(SEQ ID NO: 48)
TARGPSW
(SEQ ID NO: 49)
GGWHTGRN
(SEQ ID NO: 50)
HTGRSGAL
(SEQ ID NO: 51)
PLTGRSGG
(SEQ ID NO: 52)
LTGRSGA
SEQ ID NO: 53
matriptase cleavable peptide linker
(SEQ ID NO: 53)
RQARVVNG
SEQ ID NO: 54
matriptase-MMP2/9 dual cleavable peptide linker
(SEQ ID NO: 54)
VHMPLGFLGP RQARVVNG.
Fc with hole mutations IL-12 P35 linkerIL-12Rβ2 fragment
SEQ ID NO: 55
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGG SRNLPVATPD PGMFPCLHHS QNLLRAVSNM LQKARQTLEF
YPCTSEEIDH EDITKDKTST VEACLPLELT KNESCLNSRE TSFITNGSCL ASRKTSFMMA LCLSSIYEDL
KMYQVEFKTM NAKLLMDPKR QIFLDQNMLA VIDELMQALN FNSETVPQKS SLEEPDFYKT KIKLCILLHA
FRIRAVTIDR VMSYLNAS(G GGGS)n₁VHM PLGFLGPRQA RVVN(GGGGS )n₂KIDACKR GDVTVKPSHV
ILLGSTVNIT CSLKPRQGCF HYSRRNKLIL YKFDRRINFH HGHSLNSQVT GLPLGTTLFV CKLACINSDE
IQICGAEIFV GVAPEQPQNL SCIQKGEQGT VACTWERGRD THLYTEYTLQ LSGPKNLTWQ KQCKDIYCDY
LDFGINLTPE SPESNFTAKV TAVNSLGSSS SLPSTFTFLD IVRPLPPWDI RIKFQKASVS RCTLYWRDEG
LVLLNRLRYR PSNSRLWNMV NVTKAKGRHD LLDLKPFTEY EFQISSKLHL YKGSWSDWSE SLRAQTPEEE
PTG;
n1 = 1, 2, 3, or 4, and n2 = 1, 2, 3, or 4.
Fc with knobs mutations IL-12 P40 non-cleavable linker-IL-12Rβ1 fragment
SEQ ID NO: 56
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSIWELKKDV YVVELDWYPD APGEMVVLTC DTPEEDGITW
TLDQSSEVLG SGKTLTIQVK EFGDAGQYTC HKGGEVLSHS LLLLHKKEDG IWSTDILKDQ KEPKNKTFLR
CEAKNYSGRF TCWWLTTIST DLTFSVKSSR GSSDPQGVTC GAATLSAERV RGDNKEYEYS VECQEDSACP
AAEESLPIEV MVDAVHKLKY ENYTSSFFIR DIIKPDPPKN LQLKPLKNSR QVEVSWEYPD TWSTPHSYFS
LTFCVQVQGK SKREKKDRVF TDKTSATVIC RKNASISVRA QDRYYSSSWS EWASVPCSGG GGSGGGGSGG
GGSRTSECCF QDPPYPDADS GSASGPRDLR CYRISSDRYE CSWQYEGPTA GVSHFLRCCL SSGRCCYFAA
GSATRLQFSD QAGVSVLYTV TLWVESWARN QTEKSPEVTL QLYNSVKYEP PLGDIKVSKL AGQLRMEWET
PDNQVGAEVQ FRHRTPSSPW KLGDCGPQDD DTESCLCPLE MNVAQEFQLR RRQLGSQGSS WSKWSSPVCV
PPENPPQPQV RFSVEQLGQD GRRRLTLKEQ PTQLELPEGC QGLAPGTEVT YRLQLHMLSC PCKAKATRTL
HLGKMPYLSG AAYNVAVISS NQFGPGLNQT WHIPA
Fc with knobs mutations IL-12 P40 cleavable linker IL-12Rβ1 fragment
SEQ ID NO: 57
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSIWELKKDV YVVELDWYPD APGEMVVLTC DTPEEDGITW
TLDQSSEVLG SGKTLTIQVK EFGDAGQYTC HKGGEVLSHS LLLLHKKEDG IWSTDILKDQ KEPKNKTFLR
CEAKNYSGRF TCWWLTTIST DLTFSVKSSR GSSDPQGVTC GAATLSAERV RGDNKEYEYS VECQEDSACP
AAEESLPIEV MVDAVHKLKY ENYTSSFFIR DIIKPDPPKN LQLKPLKNSR QVEVSWEYPD TWSTPHSYFS
LTFCVQVQGK SKREKKDRVF TDKTSATVIC RKNASISVRA QDRYYSSSWS EWASVPCSGG GGSGGGGSTG
RGPSWVGGGG SGGGSRTSEC CFQDPPYPDA DSGSASGPRD LRCYRISSDR YECSWQYEGP TAGVSHFLRC
CLSSGRCCYF AAGSATRLQF SDQAGVSVLY TVTLWVESWA RNQTEKSPEV TLQLYNSVKY EPPLGDIKVS
KLAGQLRMEW ETPDNQVGAE VQFRHRTPSS PWKLGDCGPQ DDDTESCLCP LEMNVAQEFQ LRRRQLGSQG
SSWSKWSSPV CVPPENPPQP QVRFSVEQLG QDGRRRLTLK EQPTQLELPE GCQGLAPGTE VTYRLQLHML
SCPCKAKATR TLHLGKMPYL SGAAYNVAVI SSNQFGPGLN QTWHIPA
Fc with knobs mutations IL-12 P40 cleavable linker scFv
SEQ ID NO: 58
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSIWELKKDV YVVELDWYPD APGEMVVLTC DTPEEDGITW
TLDQSSEVLG SGKTLTIQVK EFGDAGQYTC HKGGEVLSHS LLLLHKKEDG IWSTDILKDQ KEPKNKTFLR
CEAKNYSGRF TCWWLTTIST DLTFSVKSSR GSSDPQGVTC GAATLSAERV RGDNKEYEYS VECQEDSACP
AAEESLPIEV MVDAVHKLKY ENYTSSFFIR DIIKPDPPKN LQLKPLKNSR QVEVSWEYPD TWSTPHSYFS
LTFCVQVQGK SKREKKDRVF TDKTSATVIC RKNASISVRA QDRYYSSSWS EWASVPCSGG GGSGGGGSTG
RGPSWVGGGG SGGGSEVQLV QSGAEVKKPG ESLKISCKGS GYSFTTYWLG WVRQMPGKGL DWIGIMSPVD
SDIRYSPSFQ GQVTMSVDKS ITTAYLQWNS LKASDTAMYY CARRRPGQGY FDFWGQGTLV TVSSGGGGSG
GGGSGGGGSD IQMTQSPSSL SASVGDRVTI TCRASQGISS WLAWYQQKPE KAPKSLIYAA SSLQSGVPSR
FSGSGSGTDF TLTISSLQPE DFATYYCQQY NIYPYTFGQG TKLEIK
Fc with knobs mutations IL-12 P40 cleavable linker scFv
SEQ ID NO: 59
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSIWELKKDV YVVELDWYPD APGEMVVLTC DTPEEDGITW
TLDQSSEVLG SGKTLTIQVK EFGDAGQYTC HKGGEVLSHS LLLLHKKEDG IWSTDILKDQ KEPKNKTFLR
CEAKNYSGRF TCWWLTTIST DLTFSVKSSR GSSDPQGVTC GAATLSAERV RGDNKEYEYS VECQEDSACP
AAEESLPIEV MVDAVHKLKY ENYTSSFFIR DIIKPDPPKN LQLKPLKNSR QVEVSWEYPD TWSTPHSYFS
LTFCVQVQGK SKREKKDRVF TDKTSATVIC RKNASISVRA QDRYYSSSWS EWASVPCSGG GGSGGGGSTG
RGPSWVGGGG SGGGSDIQMT QSPSSLSASV GDRVTITCRA SQGISSWLAW YQQKPEKAPK SLIYAASSLQ
SGVPSRFSGS GSGTDFTLTI SSLQPEDFAT YYCQQYNIYP YTFGQGTKLE IKGGGGSGGG GSGGGGSEVQ
LVQSGAEVKK PGESLKISCK GSGYSFTTYW LGWVRQMPGK GLDWIGIMSP VDSDIRYSPS FQGQVTMSVD
KSITTAYLQW NSLKASDTAM YYCARRRPGQ GYFDFWGQGT LVTVSS
Fc with hole mutations IL-12 P40 linker IL-12Rβ2 fragment
SEQ ID NO: 60
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGG SIWELKKDVY VVELDWYPDA PGEMVVLTCD TPEEDGITWT
LDQSSEVLGS GKTLTIQVKE FGDAGQYTCH KGGEVLSHSL LLLHKKEDGI WSTDILKDQK EPKNKTFLRC
EAKNYSGRFT CWWLTTISTD LTFSVKSSRG SSDPQGVTCG AATLSAERVR GDNKEYEYSV ECQEDSACPA
AEESLPIEVM VDAVHKLKYE NYTSSFFIRD IIKPDPPKNL QLKPLKNSRQ VEVSWEYPDT WSTPHSYFSL
TFCVQVQGKS KREKKDRVFT DKTSATVICR KNASISVRAQ DRYYSSSWSE WASVPCS(GG GGS) ₙ₁VHMP
LGFLGPRQAR VVN(GGGGS) ₙ₂KIDACKRG DVTVKPSHVI LLGSTVNITC SLKPRQGCFH YSRRNKLILY
KFDRRINFHH GHSLNSQVTG LPLGTTLFVC KLACINSDEI QICGAEIFVG VAPEQPQNLS CIQKGEQGTV
ACTWERGRDT HLYTEYTLQL SGPKNLTWQK QCKDIYCDYL DFGINLTPES PESNFTAKVT AVNSLGSSSS
LPSTFTFLDI VRPLPPWDIR IKFQKASVSR CTLYWRDEGL VLLNRLRYRP SNSRLWNMVN VTKAKGRHDL
LDLKPFTEYE FQISSKLHLY KGSWSDWSES LRAQTPEEEP TG; n1 = 1, 2, 3, or 4, and n2 =
1, 2, 3, or 4.
Fc with knobs mutations IL-12 P35 non-cleavable linker - IL-12Rβ1 fragment
SEQ ID NO: 61
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSRNLPVATP DPGMFPCLHH SQNLLRAVSN MLQKARQTLE
FYPCTSEEID HEDITKDKTS TVEACLPLEL TKNESCLNSR ETSFITNGSC LASRKTSFMM ALCLSSIYED
LKMYQVEFKT MNAKLLMDPK RQIFLDQNML AVIDELMQAL NFNSETVPQK SSLEEPDFYK TKIKLCILLH
AFRIRAVTID RVMSYLNASG GGGSGGGGSG GGGSRTSECC FQDPPYPDAD SGSASGPRDL RCYRISSDRY
ECSWQYEGPT AGVSHFLRCC LSSGRCCYFA AGSATRLQFS DQAGVSVLYT VTLWVESWAR NQTEKSPEVT
LQLYNSVKYE PPLGDIKVSK LAGQLRMEWE TPDNQVGAEV QFRHRTPSSP WKLGDCGPQD DDTESCLCPL
EMNVAQEFQL RRRQLGSQGS SWSKWSSPVC VPPENPPQPQ VRFSVEQLGQ DGRRRLTLKE QPTQLELPEG
CQGLAPGTEV TYRLQLHMLS CPCKAKATRT LHLGKMPYLS GAAYNVAVIS SNQFGPGLNQ TWHIPA
Fc with knobs mutations IL-12 P35 cleavable linker IL-12Rβ1 fragment
SEQ ID NO: 62
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSRNLPVATP DPGMFPCLHH SQNLLRAVSN MLQKARQTLE
FYPCTSEEID HEDITKDKTS TVEACLPLEL TKNESCLNSR ETSFITNGSC LASRKTSFMM ALCLSSIYED
LKMYQVEFKT MNAKLLMDPK RQIFLDQNML AVIDELMQAL NFNSETVPQK SSLEEPDFYK TKIKLCILLH
AFRIRAVTID RVMSYLNASG GGGSGGGGST GRGPSWVGGG GSGGGSRTSE CCFQDPPYPD ADSGSASGPR
DLRCYRISSD RYECSWQYEG PTAGVSHFLR CCLSSGRCCY FAAGSATRLQ FSDQAGVSVL YTVTLWVESW
ARNQTEKSPE VTLQLYNSVK YEPPLGDIKV SKLAGQLRME WETPDNQVGA EVQFRHRTPS SPWKLGDCGP
QDDDTESCLC PLEMNVAQEF QLRRRQLGSQ GSSWSKWSSP VCVPPENPPQ PQVRFSVEQL GQDGRRRLTL
KEQPTQLELP EGCQGLAPGT EVTYRLQLHM LSCPCKAKAT RTLHLGKMPY LSGAAYNVAV ISSNQFGPGL
NQTWHIPA
Fc with knobs mutations IL-12 P35 cleavable linker scFv
SEQ ID NO: 63
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSRNLPVATP DPGMFPCLHH SQNLLRAVSN MLQKARQTLE
FYPCTSEEID HEDITKDKTS TVEACLPLEL TKNESCLNSR ETSFITNGSC LASRKTSFMM ALCLSSIYED
LKMYQVEFKT MNAKLLMDPK RQIFLDQNML AVIDELMQAL NFNSETVPQK SSLEEPDFYK TKIKLCILLH
AFRIRAVTID RVMSYLNASG GGGSGGGGST GRGPSWVGGG GSGGGSEVQL VQSGAEVKKP GESLKISCKG
SGYSFTTYWL GWVRQMPGKG LDWIGIMSPV DSDIRYSPSF QGQVTMSVDK SITTAYLQWN SLKASDTAMY
YCARRRPGQG YFDFWGQGTL VTVSSGGGGS GGGGSGGGGS DIQMTQSPSS LSASVGDRVT ITCRASQGIS
SWLAWYQQKP EKAPKSLIYA ASSLQSGVPS RFSGSGSGTD FTLTISSLQP EDFATYYCQQ YNIYPYTFGQ
GTKLEIK
Fc with knobs mutations IL-12 P35 cleavable linker scFv
SEQ ID NO: 64
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGAGGG GSGGGGSGGG GSRNLPVATP DPGMFPCLHH SQNLLRAVSN MLQKARQTLE
FYPCTSEEID HEDITKDKTS TVEACLPLEL TKNESCLNSR ETSFITNGSC LASRKTSFMM ALCLSSIYED
LKMYQVEFKT MNAKLLMDPK RQIFLDQNML AVIDELMQAL NFNSETVPQK SSLEEPDFYK TKIKLCILLH
AFRIRAVTID RVMSYLNASG GGGSGGGGST GRGPSWVGGG GSGGGSDIQM TQSPSSLSAS VGDRVTITCR
ASQGISSWLA WYQQKPEKAP KSLIYAASSL QSGVPSRFSG SGSGTDFTLT ISSLQPEDFA TYYCQQYNIY
PYTFGQGTKL EIKGGGGSGG GGSGGGGSEV QLVQSGAEVK KPGESLKISC KGSGYSFTTY WLGWVRQMPG
KGLDWIGIMS PVDSDIRYSP SFQGQVTMSV DKSITTAYLQ WNSLKASDTA MYYCARRRPG QGYFDFWGQG
TLVTVSS
Fc IL-12 (P35-P40) with knobs mutations
SEQ ID NO: 65
DKTHTCPPCP APELLGGPSV FLFPPKPKDT LMISRTPEVT CVVVDVSHED PEVKFNWYVD GVEVHNAKTK
PREEQYASTY RVVSVLTVLH QDWLNGKEYK CKVSNKALPA PIEKTISKAK GQPREPQVYT LPPCRDELTK
NQVSLWCLVK GFYPSDIAVE WESNGQPENN YKTTPPVLDS DGSFFLYSKL TVDKSRWQQG NVFSCSVMHE
ALHNHYTQKS LSLSPGA(GG GGS)n₁RNLP VATPDPGMFP CLHHSQNLLR AVSNMLQKAR QTLEFYPCTS
EEIDHEDITK DKTSTVEACL PLELTKNESC LNSRETSFIT NGSCLASRKT SFMMALCLSS IYEDLKMYQV
EFKTMNAKLL MDPKRQIFLD QNMLAVIDEL MQALNFNSET VPQKSSLEEP DFYKTKIKLC ILLHAFRIRA
VTIDRVMSYL NAS(GGGGS) n2IWELKKDV YVVELDWYPD APGEMVVLTC DTPEEDGITW TLDQSSEVLG
SGKTLTIQVK EFGDAGQYTC HKGGEVLSHS LLLLHKKEDG IWSTDILKDQ KEPKNKTFLR CEAKNYSGRF
TCWWLTTIST DLTFSVKSSR GSSDPQGVTC GAATLSAERV RGDNKEYEYS VECQEDSACP AAEESLPIEV
MVDAVHKLKY ENYTSSFFIR DIIKPDPPKN LQLKPLKNSR QVEVSWEYPD TWSTPHSYFS LTFCVQVQGK
SKREKKDRVF TDKTSATVIC RKNASISVRA QDRYYSSSWS EWASVPCS;
wherein n1 = 2, 3, 4, or 5; and wherein n2 = 2, 3, 4, or 5.
Fc with hole mutations- IL-12Rβ2-D1D2/IL-12Rβ1-D1D2
SEQ ID NO: 66
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS KIDACKRGDV TVKPSHVILL GSTVNITCSL KPRQGCFHYS
RRNKLILYKF DRRINFHHGH SLNSQVTGLP LGTTLFVCKL ACINSDEIQI CGAEIFVGVA PEQPQNLSCI
QKGEQGTVAC TWERGRDTHL YTEYTLQLSG PKNLTWQKQC KDIYCDYLDF GINLTPESPE SNFTAKVTAV
NSLGSSSSLP STFTFLDI(G GGGS) ₙ₁VHM PLGFLGPRQA RVVN(GGGGS ) ₙ₂RTSECCF QDPPYPDADS
GSASGPRDLR CYRISSDRYE CSWQYEGPTA GVSHFLRCCL SSGRCCYFAA GSATRLQFSD QAGVSVLYTV
TLWVESWARN QTEKSPEVTL QLYNSVKYEP PLGDIKVSKL AGQLRMEWET PDNQVGAEVQ FRHRTPSSPW
KLGDCGPQDD DTESCLCPLE MNVAQEFQLR RRQLGSQGSS WSKWSSPVCV PPE;
wherein n1 = 1, 2, 3, or 4, and n2 = 1, 2, 3, or 4.
Fc with hole mutations IL-12R2-D1D2D3/IL-12R1-D1D2D3
SEQ ID NO: 67
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS KIDACKRGDV TVKPSHVILL GSTVNITCSL KPRQGCFHYS
RRNKLILYKF DRRINFHHGH SLNSQVTGLP LGTTLFVCKL ACINSDEIQI CGAEIFVGVA PEQPQNLSCI
QKGEQGTVAC TWERGRDTHL YTEYTLQLSG PKNLTWQKQC KDIYCDYLDF GINLTPESPE SNFTAKVTAV
NSLGSSSSLP STFTFLDIVR PLPPWDIRIK FQKASVSRCT LYWRDEGLVL LNRLRYRPSN SRLWNMVNVT
KAKGRHDLLD LKPFTEYEFQ ISSKLHLYKG SWSDWSESLR AQTPEEEPTG (GGGGS)n₁V HMPLGFLGPR
QARVVN (GGG GS)n₂RTSEC CFQDPPYPDA DSGSASGPRD LRCYRISSDR YECSWQYEGP TAGVSHFLRC
CLSSGRCCYF AAGSATRLQF SDQAGVSVLY TVTLWVESWA RNQTEKSPEV TLQLYNSVKY EPPLGDIKVS
KLAGQLRMEW ETPDNQVGAE VQFRHRTPSS PWKLGDCGPQ DDDTESCLCP LEMNVAQEFQ LRRRQLGSQG
SSWSKWSSPV CVPPENPPQP QVRFSVEQLG QDGRRRLTLK EQPTQLELPE GCQGLAPGTE VTYRLQLHML
SCPCKAKATR TLHLGKMPYL SGAAYNVAVI SSNQFGPGLN QTWHIPA; n1 = 1, 2, 3, or 4, and
n2 = 1, 2, 3, or 4.
Fc with hole mutations- IL-12R2-D1D2/IL-12R1-D1D2
SEQ ID NO: 68
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSKIDACKR GDVTVKPSHV ILLGSTVNIT
CSLKPRQGCF HYSRRNKLIL YKFDRRINFH HGHSLNSQVT GLPLGTTLFV CKLACINSDE IQICGAEIFV
GVAPEQPQNL SCIQKGEQGT VACTWERGRD THLYTEYTLQ LSGPKNLTWQ KQCKDIYCDY LDFGINLTPE
SPESNFTAKV TAVNSLGSSS SLPSTFTFLD I(GGGGS)n₁ VHMPLGFLGP RQARVVN(GG GGS)n₂RTSE
CCFQDPPYPD ADSGSASGPR DLRCYRISSD RYECSWQYEG PTAGVSHFLR CCLSSGRCCY FAAGSATRLQ
FSDQAGVSVL YTVTLWVESW ARNQTEKSPE VTLQLYNSVK YEPPLGDIKV SKLAGQLRME WETPDNQVGA
EVQFRHRTPS SPWKLGDCGP QDDDTESCLC PLEMNVAQEF QLRRRQLGSQ GSSWSKWSSP VCVPPE;
wherein n1 = 1, 2, 3, or 4, and n2 = 1, 2, 3, or 4.
Fc with hole mutations IL-12R2-D1D2D3/IL-12R1-D1D2D3
SEQ ID NO: 69
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSKIDACKR GDVTVKPSHV ILLGSTVNIT
CSLKPRQGCF HYSRRNKLIL YKFDRRINFH HGHSLNSQVT GLPLGTTLFV CKLACINSDE IQICGAEIFV
GVAPEQPQNL SCIQKGEQGT VACTWERGRD THLYTEYTLQ LSGPKNLTWQ KQCKDIYCDY LDFGINLTPE
SPESNFTAKV TAVNSLGSSS SLPSTFTFLD IVRPLPPWDI RIKFQKASVS RCTLYWRDEG LVLLNRLRYR
PSNSRLWNMV NVTKAKGRHD LLDLKPFTEY EFQISSKLHL YKGSWSDWSE SLRAQTPEEE PTG(GGGGS)
n₁VHMPLGFL GPRQARVVN( GGGGS)n₂RT SECCFQDPPY PDADSGSASG PRDLRCYRIS SDRYECSWQY
EGPTAGVSHF LRCCLSSGRC CYFAAGSATR LQFSDQAGVS VLYTVTLWVE SWARNQTEKS PEVTLQLYNS
VKYEPPLGDI KVSKLAGQLR MEWETPDNQV GAEVQFRHRT PSSPWKLGDC GPQDDDTESC LCPLEMNVAQ
EFQLRRRQLG SQGSSWSKWS SPVCVPPENP PQPQVRFSVE QLGQDGRRRL TLKEQPTQLE LPEGCQGLAP
GTEVTYRLQL HMLSCPCKAK ATRTLHLGKM PYLSGAAYNV AVISSNQFGP GLNQTWHIPA;
n1 = 1, 2, 3, or 4, and n2 = 1, 2, 3, or 4.
Fc with hole mutations IL-12R2-D1D2D3/ scFv
SEQ ID NO: 70
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS KIDACKRGDV TVKPSHVILL GSTVNITCSL KPRQGCFHYS
RRNKLILYKF DRRINFHHGH SLNSQVTGLP LGTTLFVCKL ACINSDEIQI CGAEIFVGVA PEQPQNLSCI
QKGEQGTVAC TWERGRDTHL YTEYTLQLSG PKNLTWQKQC KDIYCDYLDF GINLTPESPE SNFTAKVTAV
NSLGSSSSLP STFTFLDIVR PLPPWDIRIK FQKASVSRCT LYWRDEGLVL LNRLRYRPSN SRLWNMVNVT
KAKGRHDLLD LKPFTEYEFQ ISSKLHLYKG SWSDWSESLR AQTPEEEPTG (GGGGS)n₁V HMPLGFLGPR
QARVVN(GGG GS)n₂DIQMT QSPSSLSASV GDRVTITCRA SQGISSWLAW YQQKPEKAPK SLIYAASSLQ
SGVPSRFSGS GSGTDFTLTI SSLQPEDFAT YYCQQYNIYP YTFGQGTKLE IKGGGGSGGG GSGGGGSEVQ
LVQSGAEVKK PGESLKISCK GSGYSFTTYW LGWVRQMPGK GLDWIGIMSP VDSDIRYSPS FQGQVTMSVD
KSITTAYLQW NSLKASDTAM YYCARRRPGQ GYFDFWGQGT LVTVSS;
n1 = 1, 2, 3, or 4, and n2 = 1, 2, 3, or 4.
Fc with hole mutations IL-12R2-D1D2D3/scFv
SEQ ID NO: 71
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSKIDACKR GDVTVKPSHV ILLGSTVNIT
CSLKPRQGCF HYSRRNKLIL YKFDRRINFH HGHSLNSQVT GLPLGTTLFV CKLACINSDE IQICGAEIFV
GVAPEQPQNL SCIQKGEQGT VACTWERGRD THLYTEYTLQ LSGPKNLTWQ KQCKDIYCDY LDFGINLTPE
SPESNFTAKV TAVNSLGSSS SLPSTFTFLD IVRPLPPWDI RIKFQKASVS RCTLYWRDEG LVLLNRLRYR
PSNSRLWNMV NVTKAKGRHD LLDLKPFTEY EFQISSKLHL YKGSWSDWSE SLRAQTPEEE PTG(GGGGS)
n₁VHMPLGFL GPRQARVVN( GGGGS)n₂DI QMTQSPSSLS ASVGDRVTIT CRASQGISSW LAWYQQKPEK
APKSLIYAAS SLQSGVPSRF SGSGSGTDFT LTISSLQPED FATYYCQQYN IYPYTFGQGT KLEIKGGGGS
GGGGSGGGGS EVQLVQSGAE VKKPGESLKI SCKGSGYSFT TYWLGWVRQM PGKGLDWIGI MSPVDSDIRY
SPSFQGQVTM SVDKSITTAY LQWNSLKASD TAMYYCARRR PGQGYFDFWG QGTLVTVSS;
n1 = 1, 2, 3, or 4, and n2 = 1, 2, 3, or 4.
Fc with hole mutations IL-12R2-D1D2D3 scFv
SEQ ID NO: 72
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSGGGS KIDACKRGDV TVKPSHVILL GSTVNITCSL KPRQGCFHYS
RRNKLILYKF DRRINFHHGH SLNSQVTGLP LGTTLFVCKL ACINSDEIQI CGAEIFVGVA PEQPQNLSCI
QKGEQGTVAC TWERGRDTHL YTEYTLQLSG PKNLTWQKQC KDIYCDYLDF GINLTPESPE SNFTAKVTAV
NSLGSSSSLP STFTFLDIVR PLPPWDIRIK FQKASVSRCT LYWRDEGLVL LNRLRYRPSN SRLWNMVNVT
KAKGRHDLLD LKPFTEYEFQ ISSKLHLYKG SWSDWSESLR AQTPEEEPTG (GGGGS)n₁V HMPLGFLGPR
QARVVN (GGG GS)n₂EVQLV QSGAEVKKPG ESLKISCKGS GYSFTTYWLG WVRQMPGKGL DWIGIMSPVD
SDIRYSPSFQ GQVTMSVDKS ITTAYLQWNS LKASDTAMYY CARRRPGQGY FDFWGQGTLV TVSSGGGGSG
GGGSGGGGSD IQMTQSPSSL SASVGDRVTI TCRASQGISS WLAWYQQKPE KAPKSLIYAA SSLQSGVPSR
FSGSGSGTDF TLTISSLQPE DFATYYCQQY NIYPYTFGQG TKLEIK;
n1 = 1, 2, 3, or 4, and n2 = 1, 2, 3, or 4.
Fc with hole mutations IL-12R2-D1D2D3/scFv
SEQ ID NO: 73
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGGGGG SGGGGSTGRG PSWVGGGGSG GGSKIDACKR GDVTVKPSHV ILLGSTVNIT
CSLKPRQGCF HYSRRNKLIL YKFDRRINFH HGHSLNSQVT GLPLGTTLFV CKLACINSDE IQICGAEIFV
GVAPEQPQNL SCIQKGEQGT VACTWERGRD THLYTEYTLQ LSGPKNLTWQ KQCKDIYCDY LDFGINLTPE
SPESNFTAKV TAVNSLGSSS SLPSTFTFLD IVRPLPPWDI RIKFQKASVS RCTLYWRDEG LVLLNRLRYR
PSNSRLWNMV NVTKAKGRHD LLDLKPFTEY EFQISSKLHL YKGSWSDWSE SLRAQTPEEE PTG(GGGGS)
ₙ₁VHMPLGFL GPRQARVVN (GGGGS)n₂EV QLVQSGAEVK KPGESLKISC KGSGYSFTTY WLGWVRQMPG
KGLDWIGIMS PVDSDIRYSP SFQGQVTMSV DKSITTAYLQ WNSLKASDTA MYYCARRRPG QGYFDFWGQG
TLVTVSSGGG GSGGGGSGGG GSDIQMTQSP SSLSASVGDR VTITCRASQG ISSWLAWYQQ KPEKAPKSLI
YAASSLQSGV PSRFSGSGSG TDFTLTISSL QPEDFATYYC QQYNIYPYTF GQGTKLEIK; n1 = 1,
2, 3, or 4, and n2 = 1, 2, 3, or 4.
IL-2 agonist polypeptide
SEQ ID NO: 74
APXₐₐ₃SSSTKKT QLQLEHLLL Xₐₐ₂₀ LQMILNGINN YKNPKLTRML TXₐₐ₄₂KFXₐₐ₄₅MPKKA TELKHLQGLE
EELKPLEEVL NXₐₐ₇₂Xₐₐ₇₃QSKNFHL RPRDLI SXₐₐ₈₈IXₐₐ₉₀ VIVLELKGSE TTFMCEYADE TATIVEFLNR
WITFXₐₐ₁₂₅Xₐₐ₁₂₆SIIS TLT
wherein Xₐₐ₃ is N or A; wherein Xₐₐ₁₂₅ is C or S; wherein Xₐₐ₂₀ is selected
from D, H, K, L, M, N, Q, R, S, V, and Y; wherein Xₐₐ₄₂ is selected from A, G,
S, T, Q, E, N, D, R, and K; wherein Xₐₐ₄₅ is selected from A, G, S, T, Q, E, N,
D, R, and K; wherein Xₐₐ₇₂ is selected from A, G, S, T, Q, E, N, D, R, and K;
wherein Xₐₐ₇₃ is A; wherein Xₐₐ₈₈ is selected from N, A, E, F, H, K, L, M, S, T,
V, W, and Y; wherein Xₐₐ₉₀ is N or T, and wherein Xₐₐ₁₂₆ is selected from A, D, F,
G, H, I, K, L, P, S, T, W and Y; and wherein at least one of Xₐₐ₂₀, Xₐₐ₈₈,
Xₐₐ₉₀, and Xₐₐ₁₂₆ is mutated.
IL-2 agonist polypeptide
SEQ ID NO: 75
APXₐₐ₃SSSTKKT QLQLEHLLL Xₐₐ₂₀ LQMILNGINN YKNPKLTXₐₐ₃₈ML TXₐₐ₄₂KFXₐₐ₄₅MPKKA
TELKHLQGLE EXₐₐ₆₂LKPLEXₐₐ₆₈VL NLXₐₐ₇₃QSKNFHL RPRDLISXₐₐ₈₈IXₐₐ₉₀ VIVLELKGSE
TTFMCEYADE TATIVEFLNR WITFXₐₐ₁₂₅Xaa₁₂₆SIIS TLT
wherein Xₐₐ₃ is N or A; wherein Xₐₐ₁₂₅ is C or S; wherein Xₐₐ₂₀ is selected
from D, H, K, L, M, N, Q, R, S, V, and Y; wherein Xₐₐ₃₈ is selected from A, K
and S; wherein Xₐₐ₄₂ is selected from A, G, I, S, T, Q, E, N, D, R, and K;
wherein Xₐₐ₄₅ is selected from A, G, S, T, Q, E, N, D, R, and K; wherein Xₐₐ₆₂
is selected from E, L, A and I; wherein Xₐₐ₆₈ is E or V; wherein Xₐₐ₇₃ is A or
T; wherein Xₐₐ₈₈ is selected from N, A, E, F, H, K, L, M, S, T, V, W, and Y;
wherein Xₐₐ₉₀ is N or T, and wherein Xₐₐ₁₂₆ is selected from A, D, F, G, H, I, K,
L, P, S, T, W and Y; and wherein at least one of Xₐₐ₂₀, Xₐₐ₈₈, Xₐₐ₉₀, and Xₐₐ₁₂₆
is mutated.
scFv which binds to IL-12-VH-VL-21inker
SEQ ID NO: 76
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGKGGG GSGGGGSGPL GVRGGGGSEV QLVQSGAEVK KPGESLKISC KGSGYSFTTY
WLGWVRQMPG KGLDWIGIMS PVDSDIRYSP SFQGQVTMSV DKSITTAYLQ WNSLKASDTA MYYCARRRPG
QGYFDFWGQG TLVTVSSGGG GSGGGGSDIQ MTQSPSSLSA SVGDRVTITC RASQGISSWL AWYQQKPEKA
PKSLIYAASS LQSGVPSRFS GSGSGTDFTL TISSLQPEDF ATYYCQQYNI YPYTFGQGTK LEIK
scFv which binds to IL-12-VH-VL-31inker
SEQ ID NO: 77
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGKGGG GSGGGGSGPL GVRGGGGSEV QLVQSGAEVK KPGESLKISC KGSGYSFTTY
WLGWVRQMPG KGLDWIGIMS PVDSDIRYSP SFQGQVTMSV DKSITTAYLQ WNSLKASDTA MYYCARRRPG
QGYFDFWGQG TLVTVSSGGG GSGGGGSGGG GSDIQMTQSP SSLSASVGDR VTITCRASQG ISSWLAWYQQ
KPEKAPKSLI YAASSLQSGV PSRFSGSGSG TDFTLTISSL QPEDFATYYC QQYNIYPYTF GQGTKLEIK
scFv which binds to IL-12-VL-VH-31inker
SEQ ID NO: 78
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGKGGG GSGGGGSGPL GVRGGGGSDI QMTQSPSSLS ASVGDRVTIT CRASQGISSW
LAWYQQKPEK APKSLIYAAS SLQSGVPSRF SGSGSGTDFT LTISSLQPED FATYYCQQYN IYPYTFGQGT
KLEIKGGGGS GGGGSGGGGS EVQLVQSGAE VKKPGESLKI SCKGSGYSFT TYWLGWVRQM PGKGLDWIGI
MSPVDSDIRY SPSFQGQVTM SVDKSITTAY LQWNSLKASD TAMYYCARRR PGQGYFDFWG QGTLVTVSS
scFv which binds to IL-12-VL-VH-41inker
SEQ ID NO: 79
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVCT LPPSRDELTK
NQVSLSCAVK GFYPSDIAVE WESNGQPENN YKTTPPVLDS DGSFFLVSKL TVDKSRWQQG NVFSCSVMHE
ALHNHYTQKS LSLSPGKGGG GSGGGGSGPL GVRGGGGSDI QMTQSPSSLS ASVGDRVTIT CRASQGISSW
LAWYQQKPEK APKSLIYAAS SLQSGVPSRF SGSGSGTDFT LTISSLQPED FATYYCQQYN IYPYTFGQGT
KLEIKGGGGS GGGGSGGGGS GGGGSEVQLV QSGAEVKKPG ESLKISCKGS GYSFTTYWLG WVRQMPGKGL
DWIGIMSPVD SDIRYSPSFQ GQVTMSVDKS ITTAYLQWNS LKASDTAMYY CARRRPGQGY FDFWGQGTLV
TVSS
G₄S₄ linker
SEQ ID NO: 80
GGGGSGGGGS GGGGSGGGGS

1. A heterodimeric prodrug comprising an IL-12 cytokine moiety, a masking moiety (M), a dimeric carrier moiety (C), and at least one peptide linker, wherein
i) the IL-12 cytokine moiety comprises an IL-12 p40 subunit (p40) and an IL-12 p35 subunit (p35);
ii) the masking moiety binds to the IL-12 cytokine moiety and inhibits a biological activity of the IL-12 cytokine moiety; and
iii) the masking moiety is fused to one polypeptide chain of the dimeric carrier moiety, optionally via a peptide linker, and the IL-12 cytokine moiety is fused to the other polypeptide chain of the carrier moiety, optionally via a peptide linker, or
the p40 subunit is fused to one polypeptide chain of the carrier moiety, optionally via a peptide linker, the p35 subunit is fused to the other polypeptide chain of the carrier moiety, optionally via a peptide linker, and the masking moiety is fused to the C-terminus of the p40 subunit or the p35 subunit, optionally via a peptide linker.

2. The prodrug of claim 1, wherein the at least one peptide linker is a cleavable peptide linker.

3. The prodrug of claim 1 or 2, wherein the at least one peptide linker is a non-cleavable peptide linker.

4. The prodrug of any one of the preceding claims, wherein the masking moiety is a first masking moiety (MM1) that is fused to the carrier moiety or to the IL-12 p40 or p35 subunit, optionally via a cleavable peptide linker.

5. The prodrug of claim 4, wherein the first masking moiety (MM1) binds to IL-12 p40 subunit and reduces or inhibits the binding of IL-12 to IL-12 receptor β1 (IL-12Rβ1).

6. The prodrug of claim 4 or 5, wherein the first masking moiety is selected from the extracellular domain of IL-12 receptor Rβ1 (IL-12Rβ1 ECD) or a fragment thereof, a p40-binding peptide, and a single chain Fv (scFv) or Fab domain of an antibody that binds to p40.

7. The prodrug of any one of claims 4-6, wherein the first masking moiety is a scFv or Fab domain of an antibody that binds to p40.

8. The prodrug of claim 7, wherein the first masking moiety comprises a scFv that binds to p40, wherein the scFv comprises an amino acid sequence at least 99% identical to one of SEQ ID NOs: 7-11 and 12.

9. The prodrug of any one of the preceding claims, wherein the prodrug further comprises a second masking moiety (MM2) that is fused to the carrier moiety or to the IL-12 p40 or p35 subunit, optionally via a cleavable peptide linker.

10. The prodrug of claim 9, wherein the second masking moiety (MM2) binds to p35 and/or p40 and inhibits the binding of IL-12 to IL-12Rβ2.

11. The prodrug of claim 9 or 10, wherein the second masking moiety is selected from the extracellular domain of IL-12 receptor β2 (IL-12Rβ2 ECD) or a fragment thereof, a p35 or p40-binding peptide discovered from screening of a peptide library, and a single chain Fv or Fab domain of an antibody that binds to p35 or p40 and reduces or inhibits the binding of IL-12 to IL-12Rβ2.

12. The prodrug of any one of claims 9-11, wherein the second masking moiety comprises the IL-12Rβ2 ECD or a fragment thereof, wherein the IL-12Rβ2 ECD or a fragment thereof comprises an amino acid sequence at least 95% identical to SEQ ID NO: 17, 18, or 19.

13. The prodrug of any one of claims 9-11, wherein the second masking moiety comprises a single chain Fv (scFv) or Fab domain of an antibody that binds to p40 and reduces or inhibits the binding of IL-12 to IL-12Rβ2; wherein said scFv of Fab comprises the same light chain CDRs and heavy chain CDRs as derived from IL-12 antibody PMA204, Antibody 1, Antibody 50, Antibody 68, Antibody 80, or Antibody 136.

14. The prodrug of any one of the preceding claims, wherein the carrier moiety is selected from an Fc domain and an antibody.

15. The prodrug of claim 14, wherein the carrier moiety comprises a heterodimeric Fe domain comprising a first polypeptide chain and a second polypeptide chain, and wherein the polypeptide chains of the prodrug comprise molecular formulae (from N-terminus to C-terminus) selected from one of the following pairs:
a. F1-PL1-A-PL2-B, and F2-CL1-MM2-CL2-MM1; and
b. F1-PL1-B-PL2-A, and F2-CL1-MM1-CL2-MM2;
wherein F1 and F2 are the first and second polypeptide chains of the Fc domain; PL1 and PL2 are peptide linkers; CL1 and CL2 are cleavable peptide linkers; A is an IL-12 p35 subunit; and B is an IL-12 p40 subunit.

16. The prodrug of claim 14, wherein the carrier moiety comprises a heterodimeric Fc domain comprising a first polypeptide chain and a second polypeptide chain, wherein the polypeptide chains of the prodrug comprise molecular formulae (from N-terminus to C-terminus) selected from the following pairs:
a. F1-PL1-A-PL2-B, and F2-PL3-MM2-CL-MM1; and
b. F1-PL1-B-PL2-A, and F2-PL3-MM1-CL-MM2;
wherein F1 and F2 are the first and second polypeptide chains of the Fc domain; PL1, PL2, and PL3 are peptide linkers; CL is a cleavable peptide linker; A is an IL-12 p35 subunit; and B is an IL-12 p40 subunit.

17. The prodrug of claim 14, wherein the carrier moiety comprises a heterodimeric Fc domain comprising a first polypeptide chain and a second polypeptide chain, wherein the polypeptide chains of the prodrug comprise molecular formulae (from N-terminus to C-terminus) selected from one of the following pairs:
a. F1-PL1-A-CL1-MM2, and F2-PL2-B-CL2-MM1; and
b. F1-PL1-A-CL1-MM1, and F2-PL2-B-CL2-MM2;
wherein F1 and F2 are the first and second polypeptide chains of the Fc domain; PL1 and PL2 are peptide linkers; CL1 and CL2 are cleavable peptide linkers; A is an IL-12 p35 subunit; and B is an IL-12 p40 subunit.

18. The prodrug of claim 14, wherein the carrier moiety comprises a heterodimeric Fe domain comprising a first polypeptide chain and a second polypeptide chain, wherein the polypeptide chains comprise molecular formulae (from N-terminus to C-terminus) selected from one of the following pairs:
a. F1-PL1-A-CL-MM2, and F2-PL2-B-PL3-MM1; and
b. F1-PL1-A-PL2-MM1, and F2-PL3-B-CL-MM2;
wherein F1 and F2 are the first and second polypeptide chains of the Fc domain; PL1, PL2, and PL3 are peptide linkers; CL is a cleavable peptide linker; A is an IL-12 p35 subunit; and B is an IL-12 p40 subunit.

19. The prodrug of any one of claims 1-3, wherein the carrier moiety comprises a heterodimeric Fc domain comprising a first polypeptide chain and a second polypeptide chain, wherein the polypeptide chains comprise molecular formulae (from N-terminus to C-terminus) selected from one of the following pairs:
a. F1-PL1-A-PL2-B, and F2-CL-M; and
b. F1-PL1-B-PL2-A, and F2-CL-M;
wherein F1 and F2 are the first and second polypeptide chains of the Fc domain; PL1 and PL2 are peptide linkers; CL is a cleavable peptide linker; M is a masking moiety that binds to p40 subunit and inhibits a biological activity of IL-12; A is an IL-12 p35 subunit; and B is an IL-12 p40 subunit.

20. The prodrug of any one of claims 1-3, wherein the carrier moiety comprises a heterodimeric Fc domain comprising a first polypeptide chain and a second polypeptide chain, wherein the polypeptide chains comprise molecular formulae (from N-terminus to C-terminus) selected from one of the following pairs:
a. F1-PL1-A-PL2-M, and F2-PL3-B; and
b. F1-PL1-B-PL2-A, and F2-PL3-M;
wherein F1 and F2 are the first and second polypeptide chains of the Fc domain; PL1, PL2, and PL3 are peptide linkers; M is a masking moiety that binds to p40 subunit and inhibits a biological activity of IL-12; A is an IL-12 p35 subunit; and B is an IL-12 p40 subunit.

21. The prodrug of any one of claims 1-3, wherein the carrier moiety comprises a heterodimeric Fc domain comprising a first polypeptide chain and a second polypeptide chain, wherein the polypeptide chains comprise molecular formulae (from N-terminus to C-terminus) selected from one of the following pairs:
a. F1-PL1-M, and F2-PL2-A-SS-B; and
b. F1-CL, and F2-PL1-A-SS-B;
wherein F1 and F2 are the first and second polypeptide chains of the Fc domain; PL1 and PL2 are peptide linkers; M is a masking moiety that binds to p40 subunit and inhibits a biological activity of IL-12; A is an IL-12 p35 subunit; and B is an IL-12 p40 subunit; and SS is a disulfide bond.

22. The prodrug of any one of claims 19-21, wherein the masking moiety is selected from the IL-12Rβ1 ECD or a fragment thereof, a p40-binding peptide discovered from screening of a peptide library, and a single chain Fv (scFv) or Fab domain of an antibody that binds to p40.

23. The prodrug of any one of claims 19-22, wherein the masking moiety is a scFv or Fab domain of an antibody that binds to p40.

24. The prodrug of claim 23, wherein the masking moiety comprises a scFv that binds to p40, wherein the scFv comprises an amino acid sequence at least 99% identical to SEQ ID NOs: 7-11 and 12.

25. The prodrug of any one of the preceding claims, wherein the carrier is an antibody or an antigen-binding moiety that binds to a target expressed on the surface of a cancer cell.

26. The prodrug of claim 25, wherein the antibody or antigen-binding moiety binds to an antigen selected from 5T4, Claudin 18.2, EGFR, EGFR type III, GPC3, TROP-2, mesothelin, PSMA, CMET, DLL-3, and BCMA.

27. The prodrug of any one of claims 1-26, wherein the IL-12 subunit p40 has an amino acid sequence at least 95% identical to that of SEQ ID NO: 5.

28. The prodrug of any one of claims 1-27, wherein the IL-12 subunit p35 has an amino acid sequence at least 95% identical to that of SEQ ID NO: 6.

29. The prodrug of any one of claims 2-28, wherein the non-cleavable peptide linker comprises an amino acid sequence from SEQ ID NOs: 29-33; and wherein the cleavable peptide linker comprises an amino acid sequence selected from SEQ ID NOs: 34-54.

30. The prodrug of claim 1, wherein the prodrug comprises two polypeptide chains, wherein the first polypeptide chain comprises an amino acid sequence at least 99% identical to SEQ ID NO: 20; and wherein the second polypeptide chain comprises an amino acid sequence at least 99% identical to one selected from SEQ ID NOs: 21-28.

31. The prodrug of claim 1, wherein the prodrug comprises two polypeptide chains, wherein the first polypeptide chain comprises an amino acid sequence at least 99% identical to SEQ ID NO: 55; and wherein the second polypeptide chain comprises an amino acid sequence at least 99% identical to one selected from SEQ ID NOs: 56-59.

32. The prodrug of claim 1, wherein the prodrug comprises two polypeptide chains, wherein the first polypeptide chain comprises an amino acid sequence at least 99% identical to SEQ ID NO: 60; and the second polypeptide chain comprises an amino acid sequence at least 99% identical to one selected from SEQ ID NOs: 61-64.

33. The prodrug of claim 1, wherein the prodrug comprises two polypeptide chains, wherein the first polypeptide chain comprises an amino acid sequence at least 99% identical to SEQ ID NO: 65; and the second polypeptide chain comprises an amino acid sequence at least 99% identical to one selected from SEQ ID NOs: 66-73.

34. The prodrug of claim 1, wherein the prodrug comprises two polypeptide chains, wherein the first polypeptide chain comprises an amino acid sequence at least 99% identical to SEQ ID NO: 20; and said second polypeptide chain comprises an amino acid sequence at least 99% identical to one selected from SEQ ID NOs: 76-79.

35. A pharmaceutical composition comprising the prodrug of any one of claims 1-34 and a pharmaceutically acceptable excipient.

36. A polynucleotide or polynucleotides encoding the prodrug of any one of claims 1-34.

37. An expression vector or vectors comprising the polynucleotide or polynucleotides of claim 36.

38. A host cell comprising the vector(s) of claim 37.

39. The host cell of claim 38, wherein the gene(s) encoding uPA, MMP-2, MMP-9 and/or matriptase are knocked out in the cell.

40. A method of making the prodrug of any one of claims 1-34, comprising
culturing the host cell of claim 38 or 39 under conditions that allow expression of the prodrug, and
isolating the prodrug.

41. A method of treating a cancer or an infectious disease or stimulating the immune system in a patient in need thereof, comprising administering a therapeutically effective amount of the pharmaceutical composition of claim 35 to the patient.

42. A prodrug of any one of claims 1-34 for use in treating a cancer or an infectious disease or stimulating the immune system in the method of claim 41.

43. Use of a prodrug of any one of claims 1-34 for the manufacture of a medicament for treating a cancer or an infectious disease or stimulating the immune system in the method of claim 41.

44. The method of claim 41, the prodrug for use of claim 42, or the use of claim 43, wherein the cancer is selected from the group consisting of brain cancer, breast cancer, lung cancer, pancreatic cancer, esophageal cancer, medullary thyroid cancer, ovarian cancer, uterine cancer, prostate cancer, testicular cancer, colorectal cancer, and stomach cancer.

45. A method of treating cancer comprising
administering to a cancer patient in need thereof the pharmaceutical composition of claim 35 in combination with a second pharmaceutical composition,
wherein the second pharmaceutical composition comprises an active ingredient selected from a cytokine other than IL-12 or its fusion molecule, an antibody against PD-1, an antibody against PD-L1, an antibody against CTLA-4, an antibody against CD47, a PD-1 antibody-IL-2 fusion molecule, a PD-1-IL-7 fusion molecule, a PD-1 antibody-IL-15 fusion molecule, and a PD-1-IL-21 fusion molecule.

46. A prodrug of any one of claims 1-34 for use in treating cancer in the method of claim 45.

47. Use of a prodrug of any one of claims 1-34 for the manufacture of a medicament for treating cancer in the method of claim 45.

48. The method of claim 45, the prodrug for use of claim 46, or the use of claim 47, wherein the cancer is selected from glioblastoma (GBM), colon cancer, small cell lung cancer, pancreatic cancer, ovarian cancer, gastric cancer, liver cancer, triple-negative breast cancer, and multiple myeloma.

49. A pharmaceutical composition comprising the prodrug of any one of claims 1-34, a pharmaceutically acceptable excipient, and a second active ingredient selected from a cytokine other than IL-12 or a derivative therefrom, an antibody against PD-1, an antibody against PD-L1, an antibody against CTLA-4, an antibody against CD47, a PD-1 antibody-IL-15 fusion molecule, a PD-1-IL-2 fusion molecule, and a PD-1-IL-21 fusion molecule.
